(19) United States
(12) Patent Application Publication
(10) Pub. No.: US 2003/0023182 A1
(43) Pub. Date: Jan. 30, 2003

(54) Respiratory connector for respiratory gas analysis

(76) Inventors: James R. Mault, Evergreen, CO (US); Edwin M. Pearce, San Francisco, CA (US); Theodore W. Barber, Munich (DE); Timothy J. Prachar, Palo Alto, CA (US); Jeffrey C. Weintraub, Boulder Creek, CA (US); Kevin S. Nason, Menlo Park, CA (US); Craig M. Lawrence, Menlo Park, CA (US)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/205,246

(22) Filed: Jul. 25, 2002

Related U.S. Application Data
(63) Continuation-in-part of application No. 10/161,244, filed on May 31, 2002.
(60) Provisional application No. 60/308,067, filed on Jul. 26, 2001.

(51) Int. Cl. A61B 5/08

(52) U.S. Cl. 600/532

(57) ABSTRACT

An improved respiratory connector for use with a respiratory analyzer is provided. The respiratory connector includes a housing configured to be supported in contact with the subject, a flow pathway within the housing for passing the inhaled and exhaled gases therethrough and a connector port extending from the housing for connecting the respiratory connector to the respiratory analyzer. The respiratory connector also includes a usage indicating means within the housing for indicating usage of the respiratory connector to the subject. The respiratory analyzer includes a flow pathway operable to receive and pass inhaled and exhaled gases. A first end of the flow pathway is in fluid communication with the respiratory connector and a second end is in fluid communication with a source and sink for respiratory gases. A flow meter generates electrical signals as a function of the instantaneous flow volume of inhaled and exhaled gases passing through the flow pathway. A component gas concentration sensor generates electrical signals as a function of the instantaneous fraction of a predetermined component gas in the inhaled and/or exhaled gases as the gases pass through the flow pathway. A computation unit receives the electrical signals from the flow meter and the component gas concentration sensor and calculates at least one respiratory parameter for the subject as the subject breathes through the calorimeter.

### REFERENCE TO RELATED APPLICATIONS

This application is a continuation-in-part of U.S. patent application Ser. No. 10/161,244 filed May 31, 2002, and also claims priority of U.S. Provisional Patent Application No.60/308,067 filed Jul. 26,2001, both of which are incorporated herein by reference.

### FIELD OF THE INVENTION

This invention relates to a respiratory connector and more particularly to a respiratory connector for use with a respiratory analyzer.

### BACKGROUND OF THE INVENTION

Various respiratory analyzers are known in the art. One example of a respiratory analyzer is an indirect calorimeter. U.S. Pat. Nos. 4,917,108; 5,038,792; 5,178,155; 5,179,958; and 5,836,300, all to Mault, a co-inventor of the present application, are incorporated herein by reference. These patents disclose respiratory analyzers for measuring metabolism and related respiratory parameters through indirect calorimetry. These instruments generally employ flow meters which pass both the inhalations and the exhalations of a user breathing through the instrument and integrate the resulting instantaneous flow signals to determine total full flow volumes. In one embodiment, the exhaled gases generated by the user are passed through a carbon dioxide scrubber before passing through the flow meter so that the differences between the inhaled and exhaled volumes is essentially a measurement of the oxygen consumed by the lungs. In an alternative embodiment, the concentration of carbon dioxide exhaled by the user is determined by passing the exhaled volume through a capnometer and integrating that signal with the exhaled flow volume. The oxygen consumption can then be calculated as the difference between the inhaled and exhaled volumes minus the exhaled carbon dioxide volume.

The scrubber used with certain of these systems was relatively bulky and required replenishment after extended usage. The capnometers used with the instruments to measure carbon dioxide concentration had to be highly precise and accordingly expensive because any error in measurement of the carbon dioxide content of the exhalation produces a substantially higher error in the resulting determination of the oxygen content of the exhalation.

Additional approaches to indirect calorimetry and cardiac output monitoring are disclosed in Mault's co-pending applications Ser. Nos. 09/008,435; 09/191,782; PCT/US99/02448; PCT/US99/17553; PCT/US99/27297; PCT/US00/12745, each of which are incorporated herein by reference.

Respiratory analyzers, such as the indirect calorimeter, frequently include a disposable portion and a non-disposable portion. The disposable portion typically includes a part that comes in contact with the patient, and as a result is contaminated after use. For example, respiratory analyzers generally utilize a disposable respiratory connector to direct the flow of inhaled and exhaled gases through the respiratory analyzer as the subject breathes. Various types of respiratory connectors are known in the art. One example of a respiratory connector is a mouthpiece, while another example of a respiratory connector is a mask.

Improved hygiene, sanitation, and disease prevention is achievable by preventing or discouraging the reuse of a disposable part. Thus, there is a need in the art for a respiratory connector having a usage feature indicating a previous use of the respiratory connector.

### SUMMARY OF THE INVENTION

The present invention is an improved respiratory connector for use with a respiratory analyzer. The respiratory connector includes a housing configured to be supported in contact with the subject, a flow pathway within the housing for passing the inhaled and exhaled gases therethrough and a connector port extending from the housing for connecting the respiratory connector to the respiratory analyzer. The respiratory connector also includes a usage indicating means within the housing for indicating usage of the respiratory connector to the subject. The calorimeter includes a respiratory connector configured to be supported in contact with the subject so as to pass inhaled and exhaled gases as the subject breathes, a flow pathway operable to receive and pass inhaled and exhaled gases, and a hygiene barrier positioned to block a predetermined pathogen from the exhaled gases. A first end of the flow pathway is in fluid communication with the respiratory connector and a second end is in fluid communication with a source and sink for respiratory gases which may be either the ambient atmosphere, a mechanical ventilator, or other gas mixture source. A flow meter generates electrical signals as a function of the instantaneous flow volume of inhaled and exhaled gases passing through the flow pathway. A component gas concentration sensor generates electrical signals as a function of the instantaneous fraction of a predetermined component gas in the inhaled and/or exhaled gases as the gases pass through the flow pathway. A computation unit receives the electrical signals from the flow meter and the component gas concentration sensor and calculates at least one respiratory parameter for the subject as the subject breathes through the calorimeter.

One advantage of the present invention is that a respiratory connector is provided for use with a respiratory analyzer, and in particular an indirect calorimeter for measuring the metabolic rate of a subject. Another advantage of the present invention is that a respiratory connector is provided with improved hygiene, sanitation and disease transmission features. Still another advantage of the present invention is that a respiratory connector is provided with a visible indicator indicating whether the respiratory connector has already been used. A further advantage of the present invention is that a respiratory connector is provided with a physical indicator indicating whether the respiratory connector has already been used.

Other features and advantages of the present invention will be readily appreciated, as the same becomes better understood, after reading the subsequent description taken in conjunction with the accompanying drawings.

### BRIEF DESCRIPTION OF THE DRAWINGS

FIG. 1 is a perspective view of a respiratory calorimeter according to a first embodiment of the present invention with the calorimeter shown being used by a user;

FIG. 2 is a perspective view of the first embodiment of the invention;

FIG. 3 is a perspective view in exploded form of the first embodiment of the invention;

FIG. 4 is a cross-sectional view of the first embodiment of the invention, taken along lines 4-4 in FIG. 2;

FIG. 5 is a perspective view of the present invention with an alternative mouthpiece, shown with the disposable portion removed from the reusable portion;

FIG. 6 is a cross-sectional view of another embodiment of the present invention that is configured for improved sanitation;

FIG. 7 is a cross-sectional view of still another embodiment of the present invention with an alternative configuration for improved sanitation;

FIG. 8 is a perspective view in partially exploded form of a respiratory calorimeter according to the present invention and a hygiene filter module for use with the calorimeter;

FIG. 9 is a cross-sectional view of the hygiene filter module of FIG. 8;

FIG. 10 is a perspective view in partially exploded form of a respiratory calorimeter according to the present invention with an alternative embodiment of a mask incorporating a hygiene barrier;

FIG. 11 is a perspective view in exploded form of the disposable portion of the mask of FIG. 10;

FIG. 12 is a perspective view in partially exploded form of a respiratory calorimeter according to the present invention with another embodiment of a mask incorporating a hygiene barrier;

FIG. 13 is a perspective view in exploded form of the disposable portion of the mask of FIG. 12;

FIG. 14 is a cross-sectional view of a respiratory connector and respiratory analyzer with a usage indicator, according to the present invention;

FIG. 15 is a perspective view in partially exploded form of a respiratory calorimeter with a hygiene filter module and mask having a usage indicator, according to the present invention;

FIG. 16 is a cross-sectional view of the hygiene filter module of FIG. 15 with usage indicator;

FIGS. 17A-17C are sectional views of colorimetric usage indicator associated with a filter, according to the present invention;

FIG. 18 is a perspective view in partially exploded form of a respiratory calorimeter and mask with a visual usage indicator, according to the present invention;

FIG. 19 is a perspective view in exploded form of the disposable portion of the mask of FIG. 18;

FIGS. 20A-20D are sectional views of a pressure sensitive visual usage indicator, according to the present invention;

FIG. 21 is a block diagram of a usage identifying system, according to the present invention;

FIGS. 22A-22C are sectional views of physical usage indicators with a deformable element, according to the present invention;

FIGS. 23A-23C are sectional views of another example of a peelable film physical usage indicator, according to the present invention;

FIG. 24 is a sectional view of still another example of a physical usage indicator with a resilient end material, according to the present invention;

FIG. 25 is a sectional view of another example of an end tab physical usage indicator, according to the present invention;

FIG. 26 is a perspective view in partially exploded form of a respiratory calorimeter and mask with a physical usage indicator, according to the present invention;

FIGS. 27A-27B are sectional views of a yet another example of a end tab physical usage indicator, according to the present invention;

FIG. 28 is a sectional view of a detachable rim physical usage indicator, according to the present invention;

FIG. 29 is a sectional view of a further example of a deformable end physical usage indicator, according to the present invention; and

FIG. 30 is an elevational view of still a further example of a tear strip physical usage indicator, according to the present invention.

### DETAILED DESCRIPTION OF THE INVENTION

Basic Configuration of Calorimeter

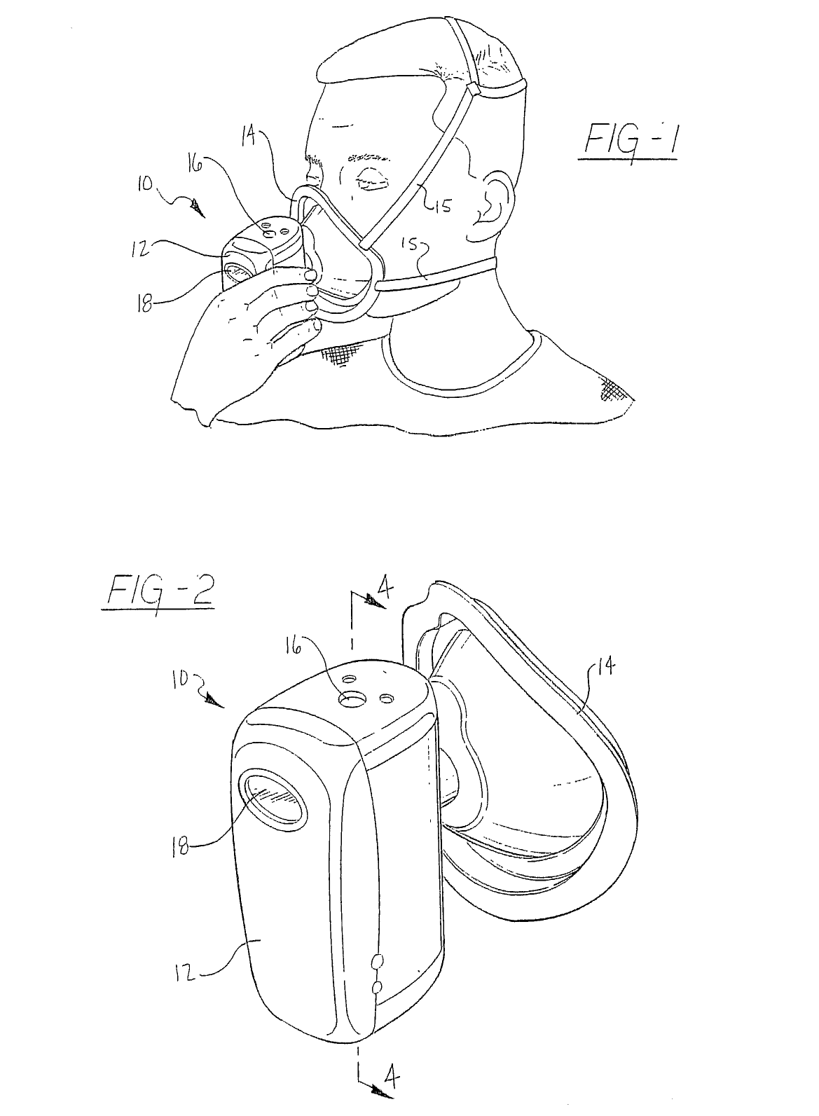

Various types of respiratory analyzers are contemplated for use with the respiratory connector of the present invention. Referring to FIGS. 1 and 2, a respiratory calorimeter is generally shown at 10. The calorimeter 10 includes a body 12 and a respiratory connector, such as mask 14, extending from the body 12. In use, the body 12 is grasped in the hand of a user and the mask 14 is brought into contact with the user's face so as to surround their mouth and nose, as best shown in FIG. 1. An optional pair of straps 15 is also shown in FIG. 1. The straps provide an alternative to holding the body 12 of the calorimeter 10 with a hand. Instead, the straps can support the mask and calorimeter in contact with the user's face.

With the mask 14 in contact with their face, the user breathes normally through the calorimeter 10 for a period of time. The calorimeter 10 measures a variety of factors and calculates one or more respiratory parameters, such as oxygen consumption and metabolic rate. A power button 16 is located on the top side of the calorimeter 10 and allows the user to control the calorimeter's functions. A separate light is located below the power button 16, with the power button 16 acting as a light pipe so that the button appears illuminated when the light is on. The light is preferably used to indicate the status of the calorimeter before, during, and after a test. A display screen is disposed behind lens 1 8 on the side of the calorimeter body 12 opposite the mask 14. Test results are displayed on the screen following a test.

Referring now to FIG. 5, a calorimeter with an alternative respiratory connector, a mouthpiece 20 rather than the mask 14 of FIG. 1, is shown. The mouthpiece 20 is preferably sized and shaped so that it may be easily inserted into a user's mouth and respiration passes through it. The mouthpiece may be made from a variety of materials, including silicone. Depending on user preference, a calorimeter according to the present invention may be used with either a mask or a mouthpiece. A mouthpiece 20 may be required for certain users, such as users with facial hair. For accurate results, it is necessary that substantially all of the user's inhalations and exhalations pass through the calorimeter. Therefore, when a mouthpiece 20 is used as a respiratory connector, it is preferred that a nose clip, not shown, be used to seal off the user's nostrils.

As best shown in FIG. 5, the body 12 of the calorimeter preferably includes a disposable flow tube portion 22 and a reusable main portion 24. The respiratory connector, such as mouthpiece 20, connects to the side of the disposable flow tube portion 22. In use, each user is given a fresh disposable portion 22 along with the appropriate respiratory connector 14 or 20. The reusable main portion may be used with multiple users. The reusable main portion 24 has a recess 26 defined in one side and shaped so as to accept the disposable portion 22.

Basic Mechanical Configuration

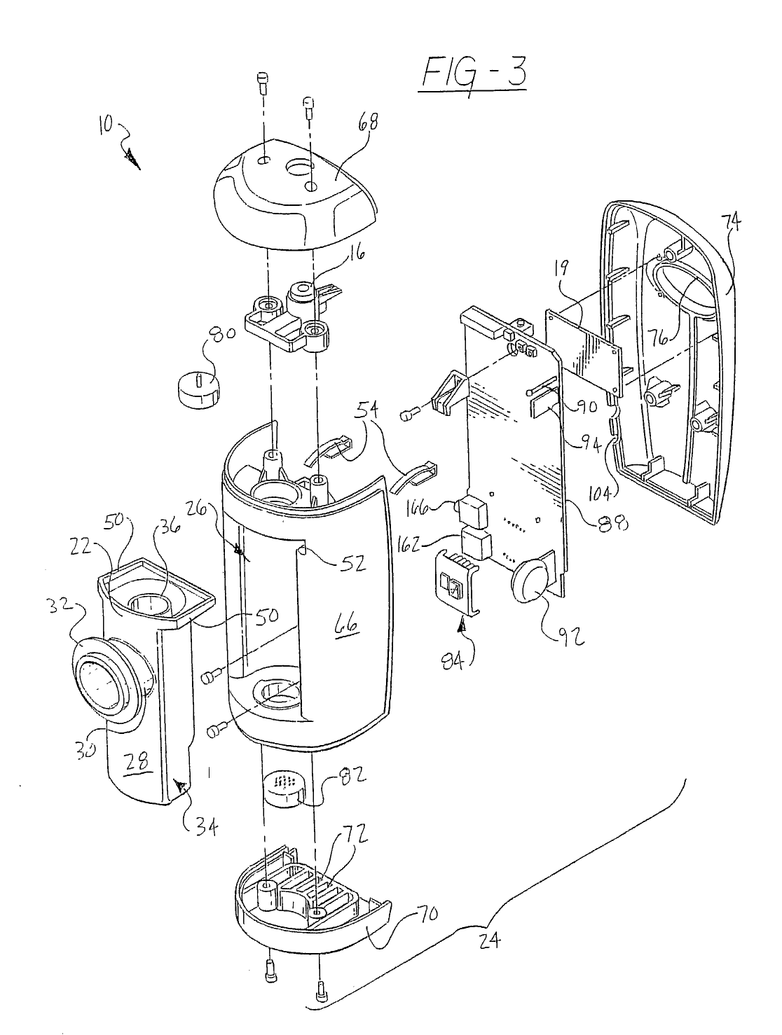

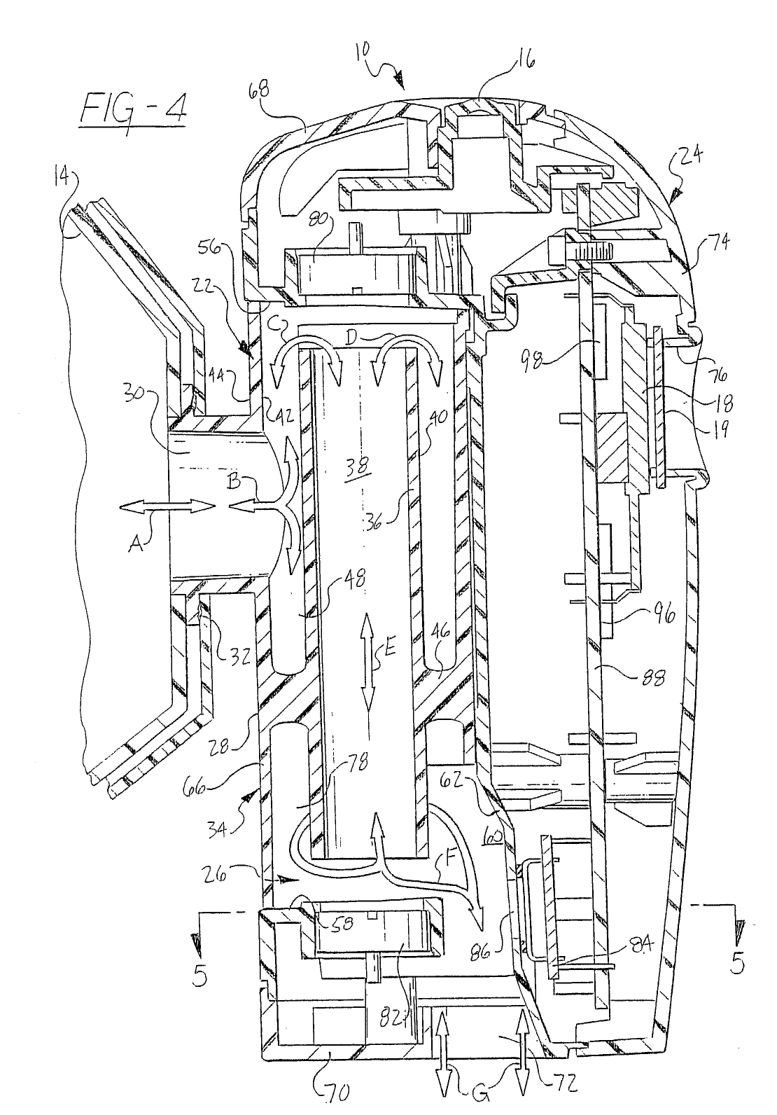

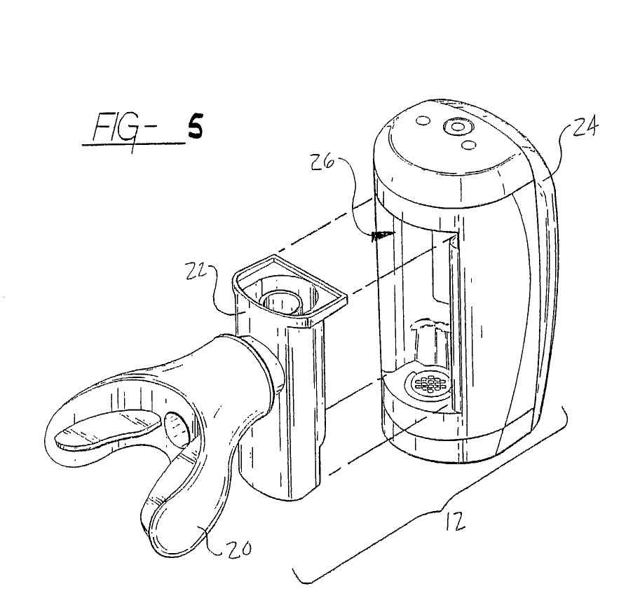

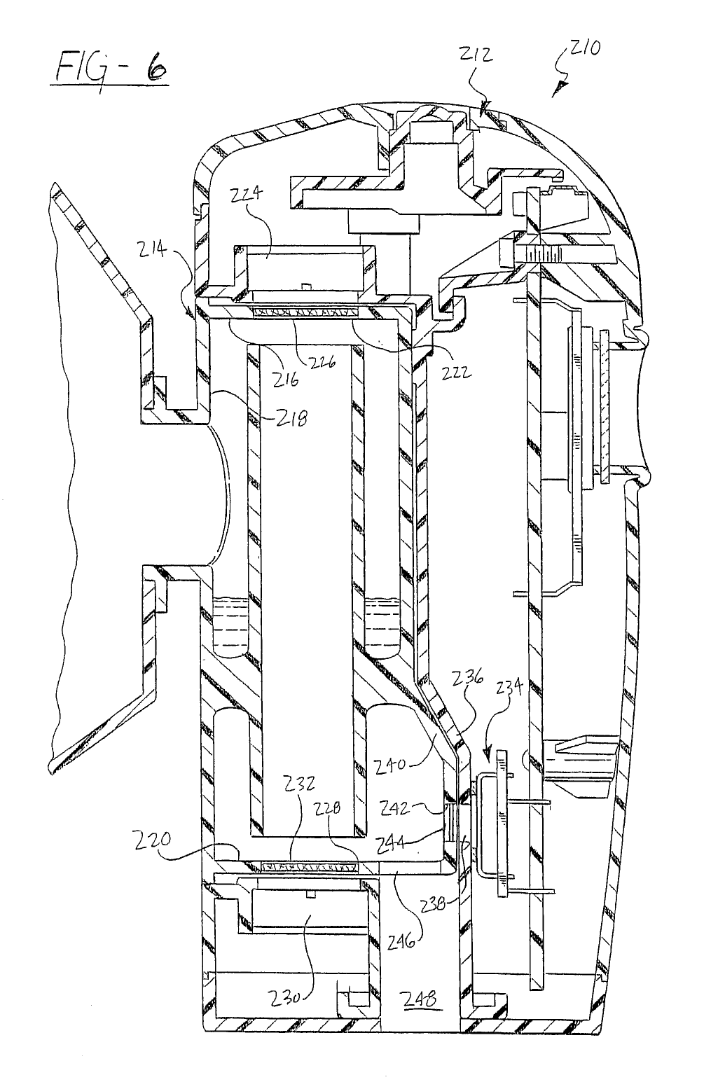

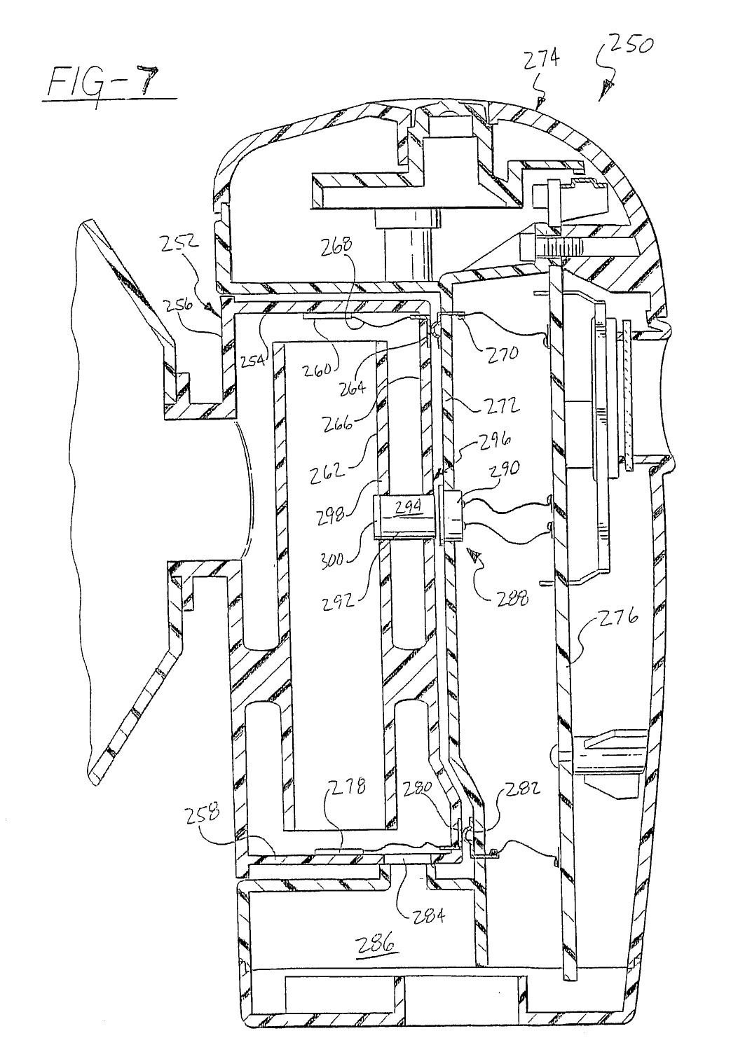

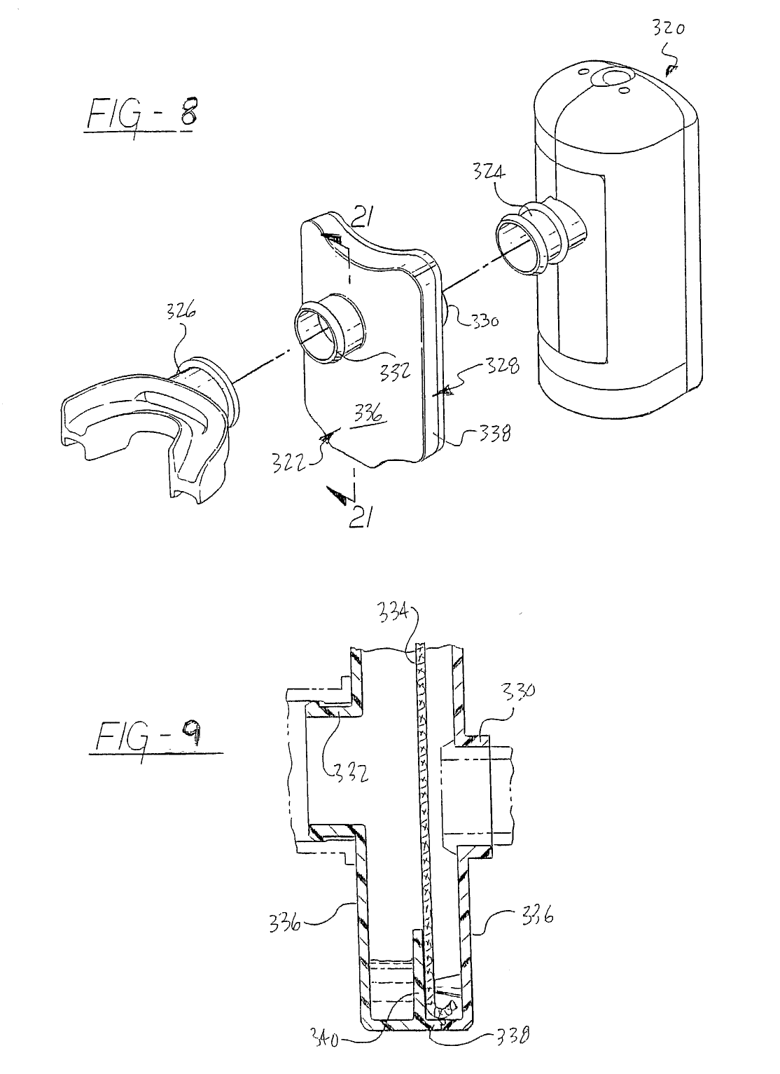

Referring now to FIGS. 3 and 4, the mechanical configuration of the calorimeter 10 will be described in more detail. FIG. 3 illustrates all components of the calorimeter in exploded form, with the disposable portion 22 removed from the recess 26 in the main portion 24. FIG. 4 is a vertical cross section of the assembled calorimeter with the disposable portion 22 docked in the main portion. Orientations such as vertical and horizontal are used throughout this specification. However, it should be understood that these orientation descriptors are used merely for convenience and are arbitrary since the calorimeter could be described in other positions.

The disposable portion 22 of the calorimeter 10 is generally elongated in the vertical direction and may be said to have a generally vertical outward face 28 which remains exposed when the disposable portion 22 is received in the recess 26. In the preferred embodiment, the outward face has a height of about 75 mm and a width of about 28 mm. An inlet conduit 30 extends perpendicularly outwardly from this outward face 28. In the preferred embodiment, the conduit 30 extends about 2 mm from the outward face 28 and has an internal diameter of about 19 mm. A radial attachment flange 32 is provided adjacent the outer end of the inlet conduit 30 and provides for attachment of a respiratory connector, such as mask 14, as best shown in FIG. 4. The respiratory connector is preferably securely attached and sealed to the attachment flange 32 such as by sonic welding.

The disposable portion 22 generally consists of an outer shell 34 with generally vertical side walls and a vertical flow tube 36 within the shell 34. The flow tube 36 is preferably cylindrical with open upper and lower ends. In the preferred embodiment, the flow tube has a length of about 63 mm and an internal diameter of about 12 mm. For definitional purposes, the flow tube 36 may be said to have an inner surface 38 on the inside of the tube 36 and an outer surface 40 on the outside of the tube 36. Likewise, the outer shell 34 may be said to have an inner surface 42 inside the shell and an outer surface 44 outside the shell. As best shown in FIG. 4, the outer surface 40 of the flow tube 36 is spaced from the inner surface 42 of the outer shell 34 so as to define a concentric gap between these two components of the disposable portion 22. The gap varies in width somewhat at different positions around the tube. However, the gap is generally at least 5 mm in width at the top of the flow tube 36, with the outer surface 40 of the tube 36 and the inner surface 42 of the shell 34 drafting toward each other slightly, for molding purposes, as the gap extends downwardly.

The flow tube 36 and the outer shell 34 are interconnected by an annular flange 46 which extends between the inner surface 42 of the outer shell 34 and the outer surface 40 of the flow tube 36. The annular flange 46 interconnects the flow tube 36 and outer shell 34 and is positioned closer to the bottom of the flow tube 36 than to the top. In the preferred embodiment, the flange 46 is positioned about 43 mm from the top of the tube 36. The flange 46 completely seals the outer surface 40 of the flow tube 36 to the inner surface 42 of the outer shell 34 so as to define a concentric chamber 48 above the flange 46 and between the outer surface 40 of the flow tube 36 and inner surface 42 of the outer shell 34.

As best shown in FIG. 4, the inlet conduit 30 is in fluid communication with the concentric chamber 48 as it intersects and penetrates the outward face 28 of the outer shell 34 above the flange 46. In the preferred embodiment, the center of the inlet conduit 30 is about 25 mm from the top of the outward face.

Referring again to FIG. 3, the upper end of the outer shell 34 of the disposable 22 has a pair of sidewardly projecting, generally horizontal, engagement rails 50. The recess 26 in the reusable portion 24 of the calorimeter has a pair of corresponding engagement slots 52, only one of which is shown. When the disposable portion 22 docks into the recess 26 of the reusable portion 24, the engagement rails 50 slide into the engagement slots 52 to securely interconnect the disposable portion and the remainder of the calorimeter 10. Springs 54 form part of the engagement slots 52 and push upwardly on the underside of the engagement rails 50. As will be clear to those of skill in the art, the disposable portion may be made from a variety of materials. In the preferred embodiment, the disposable is molded from ABS plastic.

According to one embodiment of the present invention, the disposable portion 22 and reusable portion 24 are designed such that only specifically designed authentic disposable portions work with the reusable portion. Various approaches to accomplishing this will be apparent to those of skill in the art. For example, the disposable portion may include an authenticating device such as a chip or magnetic strip that is recognized by the reusable main portion. Preferably, the calorimeter is operable only when an authentic disposable portion is docked in the reusable portion. Also, the main portion may include some type of interlock that physically “recognizes” that a correct disposable is completely docked, so that a test may not be performed with a disposable that is incorrectly or incompletely docked. As a further alternative, the reusable portion may recognize, record, and/or transmit some type of identification code associated with each disposable portion. This allows accurate record keeping. Also, specific codes can be assigned to specific users, allowing the reusable portion to identify particular users based on the disposable portion being docked.

Referring now to both FIGS. 3 and 4, the upper end of the recess 26 in the reusable main portion 34 is defined by an upper wall 56. The upper edge of the outer shell 34 of the disposable portion 22 fits against this upper wall 56 and is held in place by the springs 54. A bottom ledge 58 generally defines the lower end of the recess 26. The lower end of the outer shell 28 of the disposable portion 22 fits against this bottom ledge 58. Therefore, the upper wall 56 of the recess 26 generally seals off the upper end of the outer shell 34 of the disposable portion 22 when the disposable portion is docked with the reusable portion. Alternatively, a seal may be provided on the upper edge of the outer shell 28 or on the upper wall 56 to improve sealing. Preferably, the sides of the disposable portion 22 also fit snugly against the sides of the recess 26. It is preferred that when the disposable portion 22 is docked into the reusable portion, very little or no respiration gases passing through the disposable portion leak through the joints between the disposable portion 22 and the remainder of the calorimeter 10.

The bottom of the recess 26 is only partially defined by the bottom ledge 58. Behind the ledge 58 is an outlet flow passage 60 defined between the rear edge of the ledge 58 and the rear wall 62 of the recess 26.

The flow tube 36 does not extend as far, either upwardly or downwardly, as the outer shell 34 of the disposable portion 22. The upper end of the flow tube 36 stops short of the upper end of the outer housing and also stops short of the upper wall 56 of the recess 26 when the disposable portion 22 is docked with the reusable portion. In the preferred embodiment, a gap of about 6 mm is left between the upper end of the flow tube and the upper wall 56. Therefore, the inside of the flow tube 36 is in fluid communication with the concentric chamber 48 when the disposable portion 22 is docked in the reusable portion 24. The bottom end of the flow tube 36 also stops short of the bottom ledge 58 of the recess 26. In the preferred embodiment, a gap of about 6 mm is left between the bottom end of the flow tube and the ledge 58. Therefore, the bottom end of the flow tube 36 is not blocked off by the ledge 58 and the inside of the flow tube 36 is in fluid communication with the outlet flow passage 60 behind the ledge 58.

Referring to both FIGS. 3 and 4, the reusable main portion 24 of the calorimeter 10 has an outer housing 64 constructed from multiple pieces. A semi-cylindrical main housing member 66 defines the side walls of the reusable portion and the recess 26. A top cap 68 closes off the top of the main housing member 66 and houses the power button 16. A ventilated bottom cap 70 closes off the bottom of the main housing member 66. The bottom cap 70 includes an open grill 72 which is in fluid communication with the outlet flow passage 60 within the housing. Therefore, respiration gases and atmospheric air can flow between the area outside the calorimeter 10 and the area inside the calorimeter by flowing through the grill 72. A front cap 74 closes off the front of the main housing member 66, with front being defined as the side of the calorimeter facing away from the mask. The front cap 74 houses the lens 19 and has an oval opening 76 defined therein to allow viewing of the display screen 18 behind the lens 19. As shown, the main housing member 66, the top cap 68, the bottom cap 70, and the front cap 74 are interconnected using a variety of fasteners. Alternatively, they can be designed so as to snap together, could be adhesively interconnected, or could be interconnected in other ways. As will be clear to those of skill in the art, the components forming the outer housing 64 may be made from various materials. In the preferred embodiment, the components are molded from ABS plastic.

Approaches to Indirect Calorimetry

As is known by those of skill in the art, the above-described calorimeter provides significant packaging, air flow, and moisture removal advantages over the prior art. The actual measurements and calculations necessary to determine various respiratory and metabolic parameters may be performed in a number of ways that are known in the art. A calorimeter constructed according to the above description and accompanying figures may be configured for use with several of these approaches. Therefore, it should be understood that the following description of preferred measurement and calculation approaches is not exhaustive of the approaches possible with the physical configuration of the calorimeter thus far described.

According to a preferred embodiment of the present invention, ambient temperature, relative humidity and pressure are measured as well as inhalation volume and exhalation volume and oxygen concentration. The remaining factors are either calculated or assumed as necessary, and each of these factors may be measured in a variety of ways.

For example, there are a number of ways to determine metabolic parameters such as VO₂ (volume of oxygen consumed) and RMR (resting metabolic rate). The presently preferred approach to determining metabolic parameters uses measurements of ambient temperature, pressure and humidity along with inhalation volume, exhalation volume, and oxygen concentration in the exhalation.

VO₂, the amount of oxygen consumed, is the difference between the amount of oxygen inhaled and the amount of oxygen exhaled. It is also desirable to determine VCO₂. VCO₂ is the volume of the carbon dioxide produced by the body and is the difference between the amount of carbon dioxide exhaled and the amount of carbon dioxide inhaled. RMR may be calculated once VO₂ and VCO₂ are known. Alternatively, certain assumptions may be made concerning the ratio between VO₂ and VCO₂, allowing RMR to be calculated from VO₂ alone. Therefore, a primary purpose of the present invention is to determine VO₂. This requires determination of both the amount of the oxygen inhaled and the amount of oxygen exhaled. It is preferred to also determine VCO₂ as this allows other metabolic parameters to be determined. To determine VCO₂ requires measurement or calculation of both the amount of carbon dioxide inhaled and the amount of carbon dioxide exhaled.

Calculation of Resting Metabolic Rate

As known to those of skill in the art, resting metabolic rate (RMR) may be calculated in a variety of ways. One known and accepted approach is given by the de Weir formula, which takes the form:

RMR=1.44 (3.581×VO₂+1.448×VCO₂)−17.73

where VO₂ is the volume of oxygen consumed in milliliters-per-minute, VCO₂ is the amount of CO₂ produced in milliliters-per-minute, and RMR is the resting metabolic rate in Kcal per day. As an alternative, certain assumptions may be made concerning the ratio between VO₂ and VCO₂. Specifically, the respiratory quotient is given by the following formula:

$$R Q = \frac{V C {O}_{2}}{V {O}_{2}}$$

where RQ represents respiratory quotient. The respiratory quotient typically ranges between 0.7 and 1.1 depending on the type of stored energy source being metabolized by the user's body. RQ may be assumed to be 0.85 for typical users during the calculation of resting metabolic rate. Therefore, using this ratio and substituting for VCO₂ gives the equation:

RMR=6.929×VO₂−17.73

where RMR is resting metabolic rate in Kcal per day, and VO₂ is the volume of oxygen consumed by the user in milliliters-per-minute. Preferably, the various parameters which are measured by the calorimeter are summed or averaged over multiple breaths, thereby giving improved accuracy.

As an alternative, a CO₂ sensor may be incorporated into the calorimeter so as to directly measure, rather than calculate, CO₂ concentrations. This allows more accurate calculations of RMR as well as calculation of RQ.

Use of the Calorimeter

When the calorimeter is first turned on, the unit goes through a warm up and calibration period. During this time, the oxygen sensor heater is turned on and warms the oxygen sensor to a steady state value. During this time, the oxygen sensor is also turned on. Once the oxygen sensor has reached steady state, a zero-flow test is performed. During the zero-flow test, the flow sensor measures flow speed through the flow tube. Since the calorimeter is not being used at this stage, there should be zero flow through the flow meter. However, if the flow meter indicates a slight flow in one direction or another, an offset is assigned to reestablish zero. A variety of approaches to this zeroing may be used, though it is preferred that multiple readings are taken prior to application of an offset factor. Also, during an actual test, the flow meters may be dynamically re-zeroed during known periods of zero flow.

To use the calorimeter to calculate a subject's resting metabolic rate (RMR), it is preferred that the subject sit or relax in a comfortable position and then bring the respiratory connector into contact with their face or mouth, after the calorimeter has been turned on and allowed to warm up and self-calibrate, as previously described. The subject then breathes normally through the calorimeter for a period of several minutes. Typically, users require some amount of time before their breathing and measured metabolic rate stabilizes. Therefore, it is preferred that initial data not be used as an indication of resting metabolic rate. As will be clear to those of skill in the art, there are a variety of approaches which allow the calorimeter to most accurately determine resting metabolic rate. According to one preferred approach, once the calorimeter detects breath flow through the calorimeter, it waits 30 seconds then begins recording. However, this period of time may be increased or decreased. Once recording begins, the calorimeter makes measurements of flow, oxygen concentration, and speed of sound. Oxygen partial pressure is measured every tenth of a second, and flow velocity and speed of sound are measured 200 times per second. Flow velocity and speed of sound measurements are averaged so as to obtain a value every tenth of a second for computation of volumes. The calorimeter accumulates this data to calculate volume inspired, volume expired, inspired oxygen concentration (for calibration purposes), expired oxygen concentration, ambient temperature, ambient humidity, and ambient pressure. Ten breaths are then averaged in order to obtain one breath block. At the end of each breath block, VO₂ is calculated for the block. In order to determine steady state, three blocks are checked to see whether they are within a certain percentage of each other. For example if the previous two blocks are both within 7 percent of the current block, the block is flagged as steady state. It is determined that steady state has been reached when a certain number of consecutive blocks are flagged as steady state, such as four or five breath blocks, and then VO₂ and VCO₂ are used to calculate RMR, which is displayed on the display 18. Typically, people take 8 to 10 breaths per minute so a breath block is about one minute long. Obviously, the data may be processed in other ways. Also, certain error states may be indicated. For example, if breathing is occurring too rapidly or too slowly, an error signal may be indicated. Also, errors may be indicated for too high of a flow rate, an RMR that is out of an acceptable range, for hardware errors, or for other reasons.

As mentioned previously, it takes most users some time to stabilize their breathing and indicated rested metabolic rate. However, according to another aspect of the present invention, data during the “settling down period” may be used to predict the data during the steady state period.

A person should be fully relaxed for the measured metabolic rate to be the resting metabolic rate. However, the person's breathing will often be affected by the presence of the mouthpiece or mask, particularly during the time immediately following placing the mask over the person's nose and mouth. Accurate measurements may be delayed a certain time period, e.g. 2 minutes, after the mouthpiece has been put in place, after which the person's breathing may return to normal. However, the person may not feel comfortable with the mouthpiece in place for so long.

In order to reduce the time necessary to determine an accurate value of metabolic rate of a person, algorithms may be used to extract a resting level of VO₂ from data that is tending towards the resting value.

For a person breathing through the calorimeter of the present invention, the data can be stored by the calorimeter, and then transmitted to another electronic device for display, analysis, etc. Data may also be transmitted to another electronic device while the test is in progress (i.e. in “real time”). Data transfer from the calorimeter to another device may use flash cards (memory cards), wireless transmission (e.g. Bluetooth), cables, IR transmission, or other electromagnetic or electrical methods, or by plugging the calorimeter into the other device. The use of flash cards is disclosed more fully in Mault's provisional patent application Ser. No. 60/177,009 filed Jan. 19, 2000, and incorporated herein by reference. The calorimeter may further comprise computing means for performing data analysis.

Under certain circumstances, a user may never reach steady state during a test. Under these circumstances, the calorimeter may indicate that no reading was possible, or a steady state value may be estimated. According to one approach, the breath blocks during the test may be averaged with some additional weighting given to blocks towards the end of the test when it is assumed that the user is closer to steady state. Obviously, detailed data recorded by the calorimeter may be observed by an experienced professional to determine the reliability of the data. For example, the calorimeter may be interconnected with a desktop computer which records and/or displays data on a measurement-by-measurement or breath-by-breath basis. In this way, the professional may observe that the subject is having trouble reaching steady state and may provide counseling or suggestions on how to better interact with the device. Also, the detailed data may provide other valuable indications about the subject.

Calorimeter Embodiments with Improved Hygiene

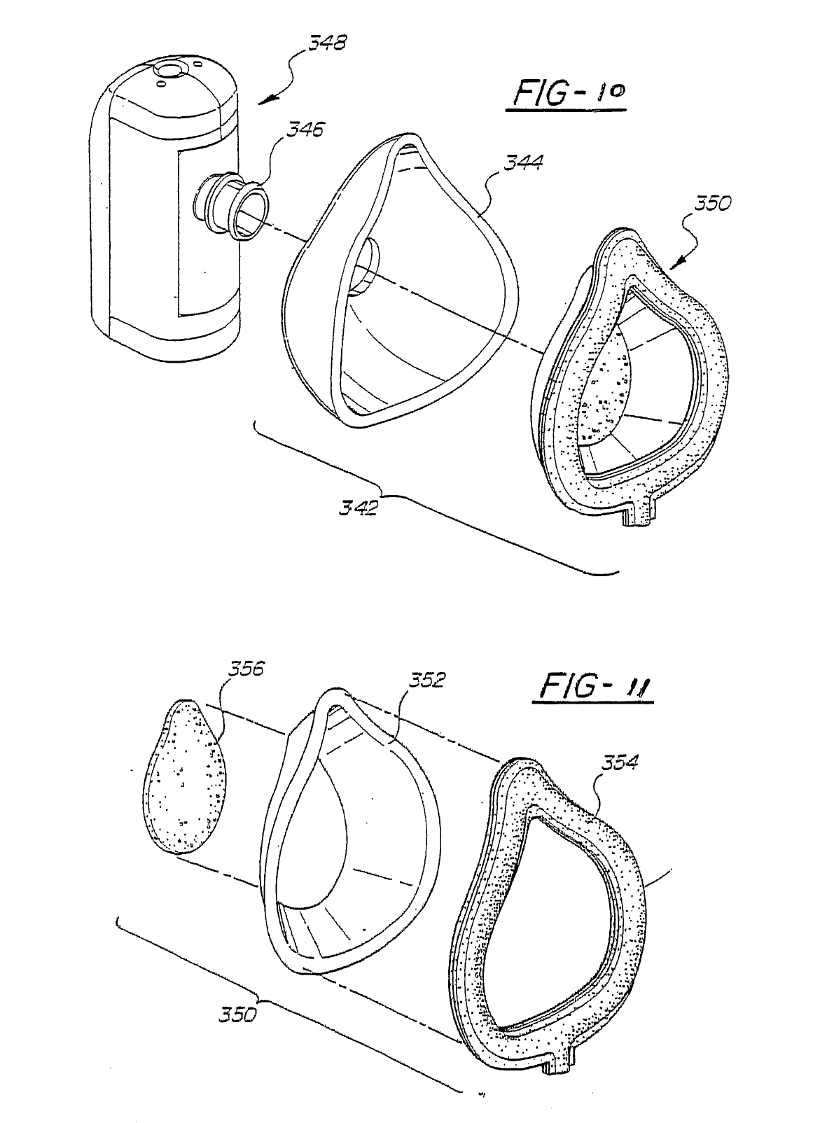

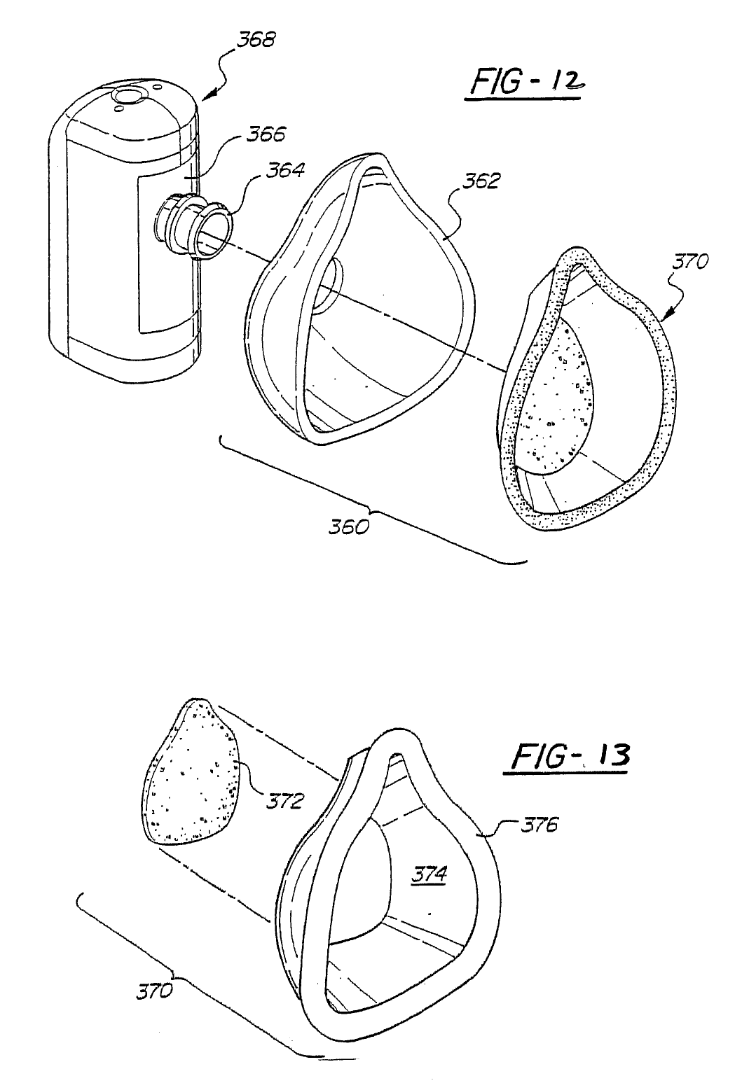

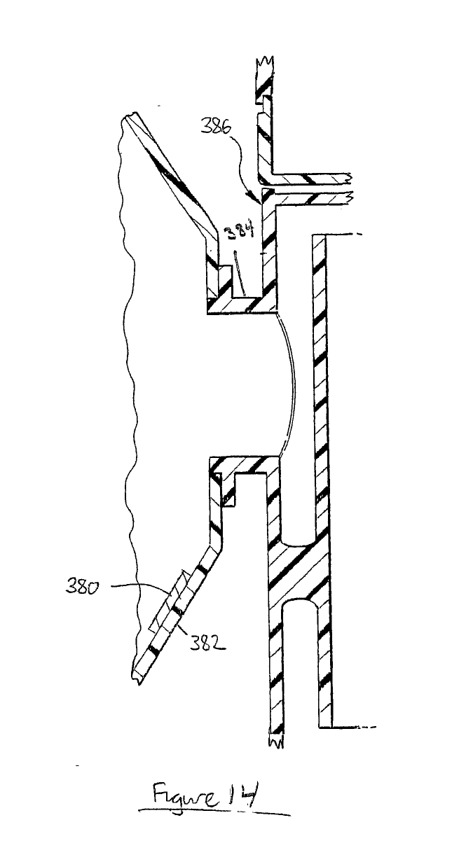

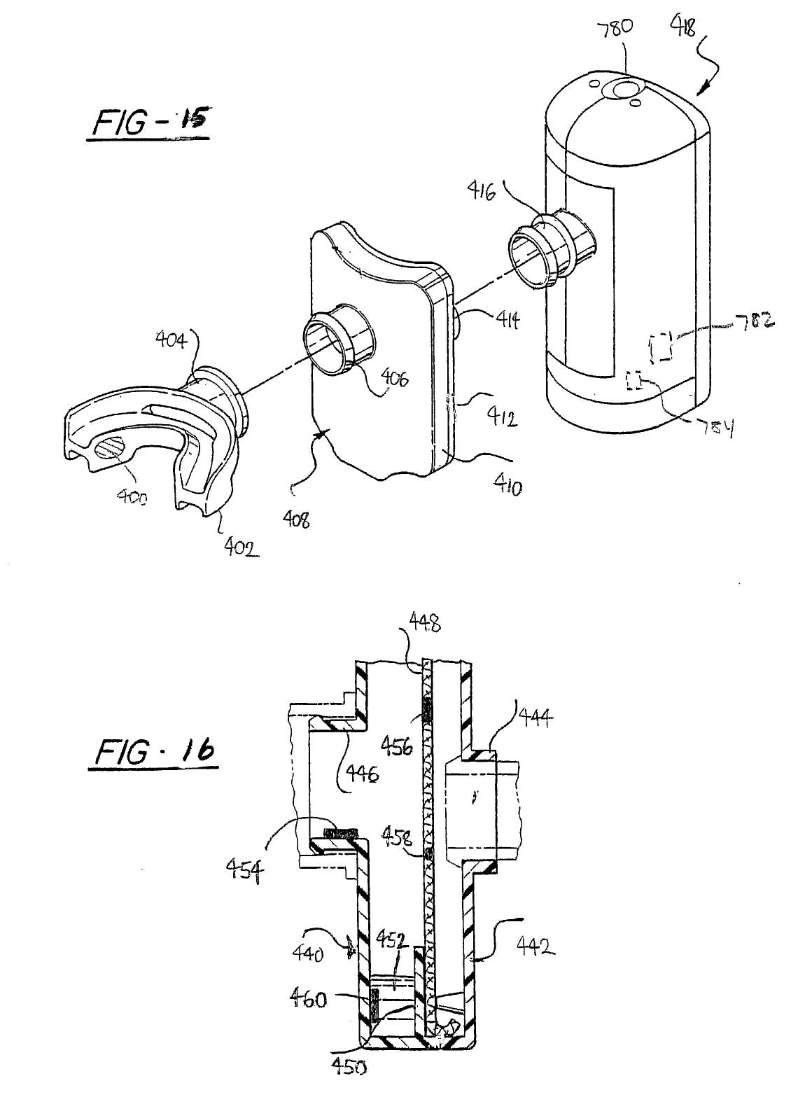

It is preferred that a calorimeter according to the present invention be able to safely be used by multiple users without undue risk of transferring pathogens from one user to another. In the previously discussed preferred embodiment of the present invention, each individual user is given their own disposable portion along with its respiratory connector. A fitness facility or a doctor may then own the reusable portion. As an alternative, each individual user may own a complete calorimeter and the disposable may merely be removable for cleaning purposes. However, it is preferred that the calorimeter be designed such that pathogens are not easily transferred from one user to another. Several improved sanitation versions of the present invention are disclosed in FIGS. 6-13.

Referring first to FIG. 6, a calorimeter according to the present invention is generally shown at 210. This calorimeter has a reusable main portion 212 that is similar to the reusable main portion 24 discussed earlier. However, in the embodiment shown in FIGS. 3 and 4, the user's inhalation and exhalations may come in contact with the ultrasonic transducers 80 and 82, the oxygen sensor 84, and the surfaces in the outlet flow passage 60. These form part of the reusable portion and therefore are not disposed or changed from user to user. The embodiment of FIG. 6 is altered so as to prevent contact of the user's breath with the transducers and oxygen sensor. The disposable portion 214 has a ceiling 216 closing off the upper end of outer shell 218 and a floor 220 closing off the lower end of the outer shell 218. A hole 222 in the ceiling 216 aligns with the upper ultrasonic transducer 224 and has a piece of germ barrier material 226 disposed in the hole 222. The barrier material may be any of a variety of materials that block the passage of pathogens but allows a passage of ultrasonic pulses. Likewise, a hole 228 is defined in the floor 220 that aligns with the lower ultrasonic transducer 230. A piece of germ barrier material 232 is also disposed in this hole 228. The oxygen sensor 234 in this embodiment is moved upwardly somewhat compared to the earlier disclosed embodiment. An opening 238 is formed in the back wall 236 of the recess in the main portion 212 with the opening 238 aligning with the oxygen sensor's forward sensing surface. The outer shell 218 of the disposable 214 has a rearward wall 240 extends down past this opening 238 and joins with the floor 220 of the disposable portion 214. An opening 242 is defined in this rearward wall 240 and a membrane 244 is disposed across the opening. The membrane is of the type that allows free passage of oxygen to the oxygen sensor, but does not allow passage of pathogens. A passage 246 is cut in the floor 220 of the disposable portion 214 allowing flow to pass into an outlet passage 248 defined in the reusable portion. This passageway 248 is large and has smooth sides to allow easy flow of inhalations and exhalations. The side walls of this passage 248 may be coated with an anti-bacterial and/or anti-viral substance to prevent contamination. Alternatively, the passageway may be cleaned between uses. As a further alternative, a disposable sleeve may be inserted into this passageway, which mates with the opening in the floor of the disposable portion. The sleeve would also be removed and disposed between users.

Referring now to FIG. 7, another alternative improved sanitation version of a calorimeter according to the present invention is generally shown at 250. As with the previously described version, the disposable portion 252 of the calorimeter 250 includes a ceiling 254 closing off the upper end of the outer shell 256 and a floor 258 closing off most of the lower end. In this version, a thin micromachined ultrasonic transducer 260 is mounted to the lower side of the ceiling 254 of the disposable portion 252 directly above the upper end of the flow tube 262, which forms part of the disposable portion. This thin ultrasonic transducer 260 replaces the larger ultrasonic transducers discussed in the earlier embodiments. The transducer may be a micromachined ultrasonic transducer array such as the ones produced by Sensant of San Jose, Calif.

Electrical contacts 264 are disposed in the rear wall 266 of the disposable portion 252, directly behind the transducer 260 and are electrically connected, such as by wires 268, to the transducer 260. Corresponding electrical contacts 270 are disposed on the rear wall 272 of the recess in the reusable portion 274 of the calorimeter 250 and align with the contacts 264 on the disposable portion 252. The contacts 270 on the reusable portion are in turn wired to the main circuit board 276. Therefore, once the disposable portion 252 is docked in the reusable portion of the calorimeter, the thin ultrasonic transducer 260 is in electrical communication with the main circuit board 276. However, because the thin transducer 260 and its associated wiring are mounted in the disposable portion 252, the entire transducer may be disposed along with a remainder of the disposable portion. This prevents any concerns about contact of the user's breath with the transducer. Alternatively, the disposable portion may be designed so as to be cleaned according to a specified cleaning procedure that does not harm the transducers.

A lower thin ultrasonic transducer 278 is disposed on the upper surface of the floor 258 of the disposable portion 252, aligned with a flow tube 262, and cooperates with the upper transducer 260 to measure flow through the flow tube. Like the upper transducer 260, the lower transducer 278 is wired to electrical contacts 280 that abut electrical contacts 282 disposed on the rear wall 272 of the recess. A passage 284 is defined in the floor 258 of the disposable portion 252 so as to allow inhalation and exhalation to flow in and out of the disposable portion. This passage communicates with a large flow area 286 in the bottom of the reusable portion 274 of the calorimeter. As an alternative, the entire lower portion of the reusable portion may be removed so that the passage in the floor of the disposable portion has no part of the reusable portion directly below it. In this way, inhalation and exhalation flowing through the passageway flows directly to and from the surrounding ambient air without coming into contact with any part of the reusable portion.

This embodiment of the calorimeter also uses an alternative version of an oxygen sensor 288. In this version, the LED and photodiode portions of the oxygen sensor are incorporated in a sensor package 290 disposed in the rear wall 272 of the recess approximately midway between the upper and lower ends of the recess. The remainder of the oxygen sensor 288 forms a part of the disposable portion 252 and is referred to as the fluorescence portion 292. The fluorescence portion 292 consists of a light pipe 294 extending from the rear surface 296 of the outer shell 256 adjacent the sensor package 290 into the wall 298 of the flow tube 262. The fluorescence material 300 is disposed on the end of the light pipe 294 so that it is in contact with the gases flowing through the flow tube 262. The light pipe 294 conducts light traveling to and from the fluorescence material 300. This configuration allows disposal of the portion of the oxygen sensor 288 that comes into contact with the user's breath. As shown, the fluorescence material 300 is positioned approximately midway in the flow tube 262. This provides a benefit in that the portion of the flow that is being sensed by the oxygen sensor is approximately at the midpoint of the portion of the flow that is being measured for flow speed. This allows better time correlation of the flow and oxygen concentration measurements.

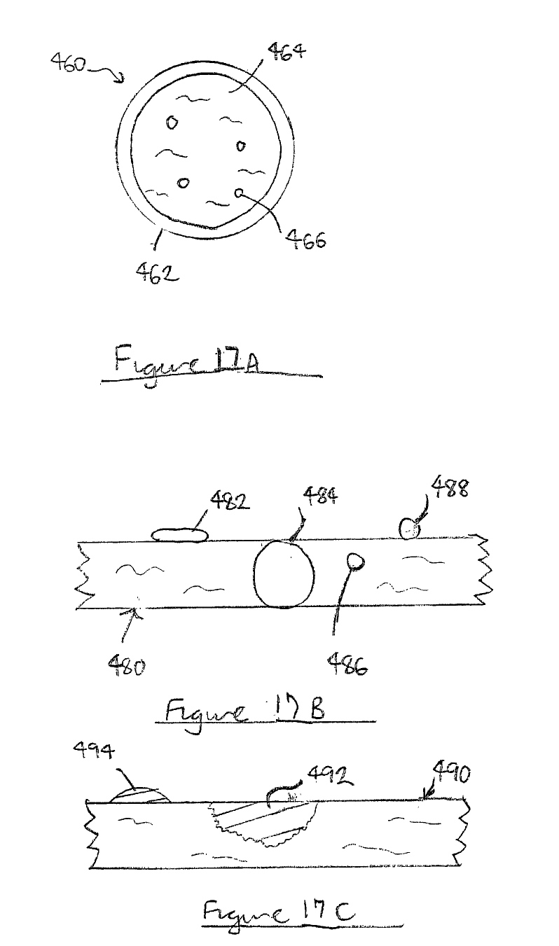

Referring now to FIG. 8, an alternative approach to improved sanitation for use with a calorimeter according to the present invention is illustrated. A calorimeter body according to any of the embodiments of the present invention is generally shown at 320. A germicidal filtration module 322 connects between the inlet conduit 324 of the calorimeter 320 and the respiratory connector, here shown as a mouthpiece 326. Referring to both FIGS. 8 and 9, the module 322 has a filter housing 328 with a calorimeter port 330 defined on one side and a respiration port 332 defined in the other. The calorimeter port 330 mates with the inlet conduit 324 of the calorimeter while the respiration port 332 mates with the respiration connector. The housing 328 may be of various shapes, including the generally rectangular configuration shown in FIG. 8. A piece of biological filter material 334, such as Filtrete® from 3M, extends within the housing 328 such that air flowing between the respiration port 332 and the calorimeter port 330 must pass through the filter material. The filter material is operable to remove pathogens thereby preventing pathogens from flowing from the respiration connector into the calorimeter. In this way, the calorimeter remains sanitary during use. Each subsequent user uses a new filter module 322 with the used module either being retained by that user or disposed.

Referring again to FIG. 9, it can be seen that the module 322 has two generally parallel and spaced apart side walls 336 with a perimeter edge 338 interconnecting the side walls 336. The filter material is generally parallel to the side walls 336 and extends between the perimeter edges 338. As best shown in FIG. 9, a saliva retention wall 340 extends upwardly from the bottom edge adjacent the filter material 334 on the side of the filter material closest to the respiration connector 326. During use of the calorimeter, especially with a mouthpiece, saliva is entrained in the exhalation breath and is preferably not introduced into the calorimeter. Much of the entrained saliva will flow along the lower edge of the respiration port 332 and down the inside of the side wall 336 where it will collect in the area between the saliva retaining wall 340 and the side wall 336, as shown. Also, some entrained saliva may contact the filter material and then fall downwardly to collect in the saliva trap. This arrangement avoids the need for the saliva trap discussed earlier in the disposable portion of the calorimeter, though it may be retained for other purposes.

Referring now to FIGS. 10 and 11, an alternative hygiene barrier arrangement is illustrated. In the configurations of FIGS. 10 and 11, a mask 342 is provided instead of a mouthpiece. In this case, the mask 342 consists of a semi-rigid outer shell 344 that interconnects with the inlet conduit 346 of the calorimeter 348. The mask shell 344 may be made of any of a variety of materials, including polystyrene. The mask shell 344 is preferably ultrasonically bonded to the inlet conduit 346 of the disposable portion of the calorimeter to provide an airtight seal. A disposable mask liner 350 is inserted into the mask shell 344. The mask liner 350 includes a liner shell 352 which overlies a portion of the masked shell 344, a face seal 354 to seal the mask 342 to the face of the user, and a hygiene barrier 356 that filters all gases flowing into and out of the calorimeter. Once again, the hygiene barrier 356 may be a material such as Filtrete® by 3 M. The face seal 354 preferably is an inflated sealed film that easily forms to the shape of the user's face providing a secure seal. The face seal 354 is securely attached, such as by a cement bond, to the liner shell 352, which is preferably a vacuum formed plastic. The hygiene barrier 356 is securely interconnected with the liner shell 352 such as by an ultrasonic bond.

Referring now to FIGS. 12 and 13, an alternative filtered mask design 360 is disclosed. Similar to the previous version, a semi-rigid mask shell 362 is interconnected with the inlet conduit 364 of the disposable portion 366 of the calorimeter 368. A mask liner 370 inserts into the shell and is disposable. The mask liner 370 includes a piece of hygiene barrier material 372 such as Filtrete® which is interconnected, such as by insert molding, to a liner shell 374 which is in turn molded with an injection molded-type face seal 376 of elastomer material. The face seal 376 securely seals to the face of the user thereby preventing leakage.

Because users vary in the size and shape of their face, mask shells and/or mask liners may be provided in a variety of sizes and shapes to suit various users. Also, as will be clear to those of skill in the art, other designs of masks and filter housings may also be used wherein the breath is filtered. According to the present invention, it is preferred that a relatively large piece of hygiene barrier material is used so as to prevent a pressure drop across the material. In this way, the barrier material does not significantly increase the resistance of flow through the calorimeter and thereby does not cause the expenditure of additional energy during use of the calorimeter.

As an alternative, a mask according to the present invention may include a nares spreader for opening the nostrils of a user, thereby reducing the effort associated with breathing through the mask. As one approach, adhesive pads may be provided inside the nose portion of the mask. The pads are pressed into contact with the nose of the user and, when released, the mask opens the nasal passages.

Other Embodiments with Improved Hygiene

In another preferred embodiment, the respiratory connector includes a usage indicating means. The usage indicating means provides the subject with an indication of previous use of the respiratory connector. With respect to the indirect calorimeter of the present invention, knowledge of previous use of the reusable portion is valuable from a sanitation, hygiene and germ prevention perspective. It is contemplated that usage of the respiratory connector is limited to a predetermined number of uses, such as one. Various embodiments of a usage indicating means are disclosed with respect to FIGS. 14-30. Depending on the use circumstances, it is possible that one or more usage indicating means can be utilized at the same time.

Various types of usage indicating means are contemplated. Examples of a usage indicating means include a visual usage indicating means, a physical usage indicating means and a usage identifying indicating means. The visual indicating means provides a visual signal to the subject regarding the condition of the respiratory connector, i.e. new or used. The physical usage indicating means is a physical signal to the subject of the condition of the respiratory connector. The usage indicating means is a usage tracking system. Advantageously, sanitation of the respiratory analyzer is improved by providing an indication of previous use of a respiratory connector, such as a mask, mouthpiece, pathogen filter, or other such replaceable element of a calorimeter.

One example of a visual usage indicating means is a colorimetric indicator that changes color when exposed to a predetermined condition, such as a component in the inhaled or exhaled gas passing through the respiratory connector. Other types of predetermined conditions are handling, exposure to air, or exposure to a fluid. The visual indicator preferably shows the subject that the replaceable component has been previously used. For example, an indicator can be one color before use, changing to another color as the subject breathes through the replaceable element. A color change occurs due to exposure of a colorimetric material to carbon dioxide, water vapor, or pathogens in the exhaled breath of a subject. The color change can occur within a geometrical shape, patch, other pattern, warning signal, message or the like. Various types of colorimetric materials are known in the art. These materials are formed into a predetermined shape, such as a color changing patch, strip, film, or other such element.

Chemical films providing a colorimetric response to exposure to carbon dioxide are known in the art. Indicators can be formed of films of such chemicals disposed on a surface of a flow pathway, mask, or mouthpiece, located so as to be exposed to exhaled gases.

Another colorimetric indicator is a colorimetric chemical that is sensitive to water vapor (moisture). Similar to a carbon dioxide indicator element, the moisture indicator element is preferably a film located on an inner surface of the flow pathway that is exposed to exhaled gases. For moisture detection, it is preferable to position the indicator element at a location where moisture accumulates during exhalations, such as lower surfaces, spit traps, or crevices, within or adjacent to filters, or other surfaces.

A further colorimetric indicator is sensitive to temperature, and changes color when exposed to a predetermined temperature. For example, if the subject exposes the respiratory connector to a predetermined temperature, such as in boiling or sterilizing or the like, then the indicator changes color to indicate previous use.

Still another colorimetric indicator is an immunological indicator element that is responsive to a predetermined pathogen in the exhaled breath of the subject.

A disposable element may be constructed in part or in full of a transparent material, such as polypropylene, so that a visual indication of previous use can be viewed through the material. For example, an internal filter may have colorimetric beads embedded in it, sensitive to carbon dioxide, which can be viewed through a transparent wall.

In U.S. Pat. No. 5,834,626, De Castro et al. describe a colorimetric indicator for moisture which may be advantageously adapted for use with embodiments of the current invention. A cobalt chloride film changes from the color blue to the color pink on exposure to moisture from exhalation. The object of the moisture indicator is to provide an indication of previous reuse. In U.S. Pat. No. 4,488,547, Mason discloses a face mask with a color indicating feature. The object of the Mason patent is to encourage replacement of a face mask after a certain period of use. In embodiments of the present invention, the indicator need not be visible to a person using the indirect calorimeter, but should be visible to a subsequent user to insure that a replaceable element for a respiratory analyzer has been replaced.

Colorimetric sensors for gas components can be advantageously combined with gas enriching polymers. For example, a carbon dioxide colorimetric indictor can be dispersed, for example as particles, side chain molecules, solid solution, or the like, in a polymer which concentrates carbon dioxide. This can increase the time period that the colorimetric indication is present. Gas concentrating polymers are disclosed in U.S. Pat. No. 5,233,194 to Mauze et al., incorporated herein by reference.

It is contemplated that surfaces, such as those of the flow path, filter elements, modules, masks, mouthpieces, or the like, can be advantageously coated with or otherwise treated with anti-pathogen coatings. Anti-pathogen coatings are disclosed in U.S. Pat. No. 6,120,784, incorporated herein by reference. It is also contemplated that respiratory connectors, including masks and mouthpieces, can also includes immunological sensors for oral bacteria, such as S. mutans.

While the visual usage indicator is preferably nonreversible, in certain examples a reversible usage indicator is advantageous. The reversible usage indicator eliminates or reverses a color change by exposure to a predetermined condition, such as a temperature. For example, a temperature sufficient to sterilize a flow pathway reverses a colorimetric indication of use. A similar approach can be used for other sterilization techniques, such as UV exposure, exposure to oxidizing chemicals, and other methods. A proprietary sterilizing solution, for example as supplied by the manufacturer of the indirect calorimeter or an affiliate, can include a chemical component to reverse a colorimetric indication of previous use.

A thermochromic film can also be used to indicate leaks, as exhaled air is typically warmer than ambient air.

Referring to FIG. 14, an example of a colorimetric indicator, which is a colorimetric indicator film 380, is illustrated. In this example, the indicator film 380 is disposed on the inside surface of the respiratory connector, which in this example is a mask 382 attached to radial attachment flange 384 of calorimeter 386. It should be appreciated that the indicator film 380 is positioned on a portion of the respiratory connector that is visible to the subject prior to use, and at the same time exposed to the flow of inhaled and exhaled air from the subject. With respect to a mask, the indicator film 380 is a patch adhered onto a portion of the mask or mask liner. It is also contemplated that the mask material include a colorimetric indicator. For example, a carbon dioxide indicator film is applied to the surface of the face seal to indicate leaks.

Referring to FIG. 15, an example of a colorimetric indicator, which is colorimetric wetness indicating film 400, is illustrated. The respiratory instrument, similar to the indirect calorimeter previously described, includes a mouthpiece 402 having a mouthpiece respiration connector port 404, and a calorimeter, generally shown at 418 having an inlet conduit 416. The calorimeter 418 also includes a germicidal filtration module 408 having a filter housing 412. The filtration module 408 further includes a calorimeter port 414, and a respiration port 406 connected between the inlet conduit 416 of the calorimeter 418 and the respiration connector port 404 of the mouthpiece 402. An indicator film 400 is disposed on a surface of the mouthpiece 402, so as to be in contact with the lips of a subject during use. The indicator film can provide a colorimetric change in response to moisture, so as to indicate previous contact with a subject's lips. A non-reversible color change is a long term indicator of previous use.

Alternatively, the plastic used to form the mouthpiece 402 includes a colorimetric material that changes its visual appearance on exposure to a component in the inhaled or exhaled breath of the subject. For example, the plastic used to form the mouthpiece can include a colorimetric indicator. Alternatively, the colorimetric indicator film is disposed on the inside surface of the mouthpiece, and is sensitive to an element in the inhaled or exhaled breath of the subject, such as carbon dioxide or moisture, or the like.

Referring to FIG. 16 an example of positioning a colorimetric indicator within the previously described filter module 440 is illustrated. The filter module 440 includes a housing 442, a calorimeter port 444, a respiration port 446, a filter material 448 and a retaining wall 450 forming a spit trap 452. It should be appreciated that one or more indicator films may be utilized. The moisture indicator film 460 is located within a spit trap 452, formed by a housing 442 and a retaining wall 450. This location is advantageous for a moisture sensitive indicator. Alternatively, an indicator film 454 is located within the respiration port 446, so as to be exposed to exhaled air. Similarly, an indicator film 458 is located within the filter material 448, so as to be exposed to exhaled air. The filter material 448 can be treated wholly or in part, so as to provide a visual indicator of moisture and/or carbon dioxide exposure. Preferably, the housing 442 is made from a transparent material, so that the subject receives a visual indicator of previous use.

Referring to FIG. 17A, an example of a colorimetric indicator within a hygiene barrier, or filter is illustrated. The filter is used in the respiratory connector or respiratory apparatus, as previously described. The filter 460 includes a support 462 surrounding a piece of filter material- 464, and colorimetric filter indicator elements 466 disposed within the filter material 464. As a person breathes through the filter 460, the colorimetric indicator elements 466 change color due to exposure to a predetermined component in the exhaled breath, such as carbon dioxide and/or moisture.

Referring to FIG. 17B, an example of a colorimetric indicator supported on a piece of filter material 480 is illustrated. It should be appreciated that the filter material 480 is attachable using a conventional attaching technique, such as an adhesive. In this example, a number of indicator elements 482 are distributed on one or both faces of the filter material, or supported within the material. Alternatively, indicator elements 484 and 486 are disposed within the filter material, or an indicator element 488 is supported on the filter material. It is contemplated that the surrounding support 462 as described with respect to FIG. 30A can be adapted to provide a visual indicator of previous use.

Referring to FIG. 17C, another example of a colorimetric indicator, such as an indicator chemical 492 dispersed throughout a part of the filter material 490, is illustrated. Droplets of indicator chemical are positioned on one or both surfaces of the film, as illustrated at 494, using a conventional technique, such as spraying. The droplets then diffuse into the film, to form regions which change color, as shown at 492.

It should be appreciated that indicator chemicals are sprayed so as to produce a plurality of droplets on a surface exposed to exhaled air. The droplets are then treated, so as to become a permanent indicator element. For example, droplets comprising a monomer and a chemical sensitive to moisture and carbon dioxide can be exposed to UV radiation, so as to produce a polymer-based indicator element.

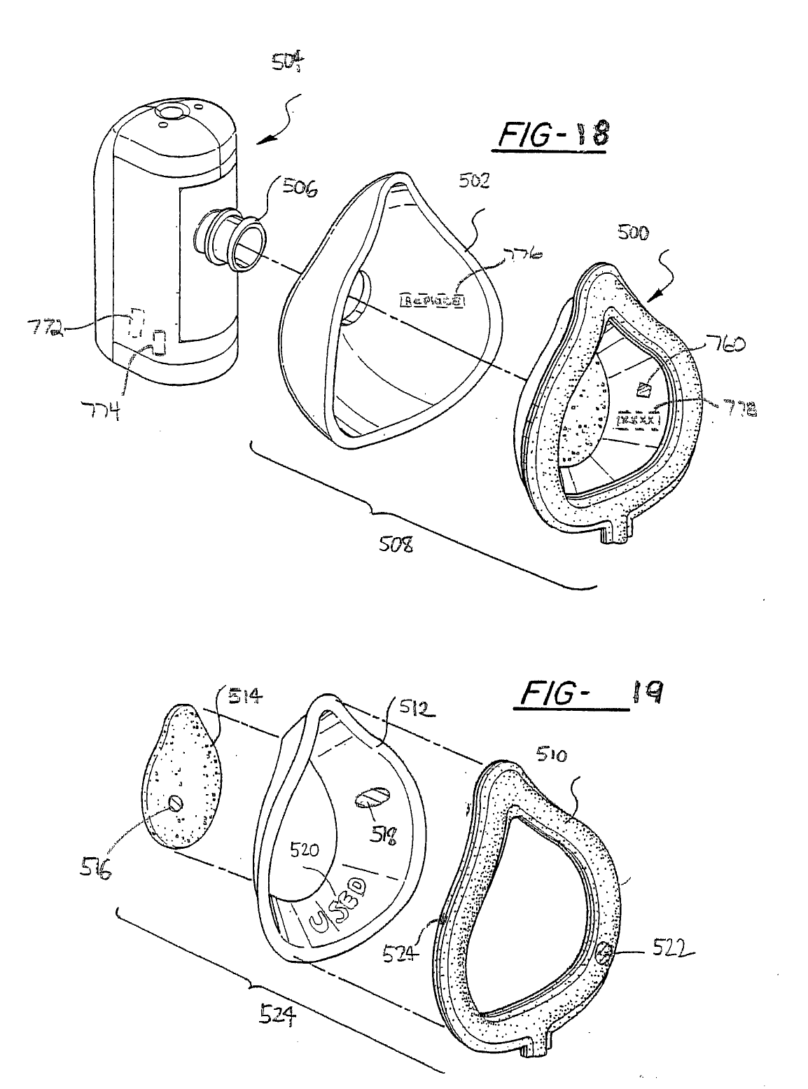

Referring to FIGS. 18 and 19, an example of an indirect calorimeter having a respiratory connector, as described with respect to FIGS. 10 and 11, with a visual usage indicator positioned in a predetermined location, is illustrated. In this example the respiratory connector is a mask. The indirect calorimeter 504 includes an inlet conduit 506 adapted to interconnect with a mask shell 502. A mask liner 500 is placed into the mask shell 502. As shown in FIG. 19, the mask liner includes a liner shell 512, a face seal 510, and a hygiene barrier 514. In one example, the hygiene barrier 514 includes a visual indicator element 516 disposed in the material. It should be appreciated that there may be a plurality of indicator elements 516 concentrated in an area, so that the hygiene barrier 514 changes color as the person breathes through the filter. In another example, the liner includes an indicator film 518 that provides a visual representation of previous use, such as spelling out the word “USED” as shown at 520. In still another example, an indicator element 522 positioned on the face seal 510 is discolored by exposure to skin oil or moisture. Alternatively, the surface of the face seal 510 is smooth, with specular reflection that is marred by contact with the skin.

In a further example, an indicator element 524 is located outside of the face seal 510. The indicator element 524 is a carbon dioxide indicator film applied to the surface of the face seal to indicate leaks. Preferably, the outside edge of the face seal is not exposed to significant concentrations of carbon dioxide. Therefore, the application of a colorimetric carbon dioxide indicator film in this region is used to locate a leak. It should be appreciated that in this example the colorimetric response is reversible; however, the typical usage indicator is nonreversible. It should be appreciated that moisture indicator films are also used in identifying leaks. For example, a thermochromic film is used to indicate a leak, as exhaled air is typically warmer than ambient air.

Referring to FIG. 20A, an example of a pressure sensitive visual usage indicator is illustrated. The pressure-induced distortion serves as a visual indicator of previous use. For example, the surface of a respiratory connector 540 includes a surface micro-relief structure 542. The surface micro-relief of this example is a molded grating structure with a grating period comparable with the wavelength of light. Other micro-relief structures may be formed, for example by stamping the surface of a face seal element which comes into contact with the skin of a person. Surface contamination, for example by fluids such as moisture and oil, change the optical properties of the surface. It should be appreciated that the micro-relief pattern is a predetermined pattern, such as lined, crosshatched, swirled, or otherwise patterned. Alternatively, the surface may be smooth, with specular reflection, which is marred by contact with the skin.

Referring to FIG. 20B, another example of a pressure sensitive visual usage indicator is illustrated. A thin deformable layer 552, with surface micro-relief 550, is supported by the surface of a face seal component 554. In this example, the layer 552 deforms under the pressure of skin contact, so as to provide a visual indicator of use.

Referring to FIG. 20C, still another example of a pressure sensitive visual usage indicator is illustrated. In this example, a thin layer of transparent material 560 is deposited on the surface of a face seal component 562. Preferably, the thin layer 560 is of a thickness which induces visible optical interference effects. Surface contamination, e.g. by films or oil, shown at 564, modify the visible appearance of the film, to indicate previous use.

Referring to FIG. 20D, a further example of a pressure sensitive visual usage indicator is illustrated. For example, the usage indicator is a thin film 574, such as a transparent plastic, supported by deformable elements 572. Preferably, the deformable elements 572 are spaced apart from the face seal component 570. The pressure applied while using the face seal will deform the elements 572, modifying the spacing as shown at 576 between the thin film 574 and face seal component 570, to change the visual appearance of the face seal.

Another example of a usage indicating means is a usage identifying indicating means. This approach is particularly useful where a single calorimeter is used by a number of users within a restricted location, such as a health club. It is assumed that a disposable component is used frequently as part of a weight or fitness control system. A separate computer is used to receive information from a person, such as identity, password, and other data. Before a metabolic measurement is performed using the indirect calorimeter, the person is requested to use a new disposable component. Preferably, as part of a licensing agreement, the health club is charged a fee for each disposable component used (e.g. mask, mouthpiece, filter holders, filters, flow pathways, and other components). The number of disposable components used is calculated from the number of tests performed using the respiratory analyzer, so that it is in the financial interest of the health club to encourage the purchase of a new disposable prior to each test. A person can enter a product code for a disposable component, for example using manual entry, barcode readers and the like, into the computer or into a respiratory analyzer. A software program then analyzes the entered product code, establishes the acceptability of the code (for example using internal check digits, or checking a database of available and/or previously used codes) before allowing the test to proceed.

Referring to FIG. 21, an example of a usage identity system is illustrated. The system includes a computer 600 in communication with a disposable product code database 610 and a health club database 608. It should be appreciated that the databases may be combinable into a single database. The computer is further in communication with an indirect calorimeter 602, a data input mechanism 604 such as a keyboard or mouse, and a display 606, such as a monitor. In use, the person to be tested is handed one or more disposable components on entering the location of the indirect calorimeter system. The person enters their personal identity data using the data input mechanism 604, and is prompted to enter a disposable product code. The entered code is checked for validity and acceptability against the product code database 610. If the product code is found to be satisfactory, the computer initiates a metabolic respiratory test, and stores the data relating to the person such as metabolic rate, in the health database 608. The user is automatically billed for the disposable components and the test. Preferably, the non-disposable part measures the number of measurements made, and this number is compared to the number of disposable parts used. For example, in a licensing arrangement, a licensed user can be billed for a number of disposable parts consistent with the number of measurements.

A further example of a usage indicating means is a physical use indicating means that prevents or discourages reuse of the respiratory connector. Examples of physical usage indicator elements include tear-away tabs, distorting components, fragile or tearable elements or other such techniques contemplated to prevent or discourage reuse.

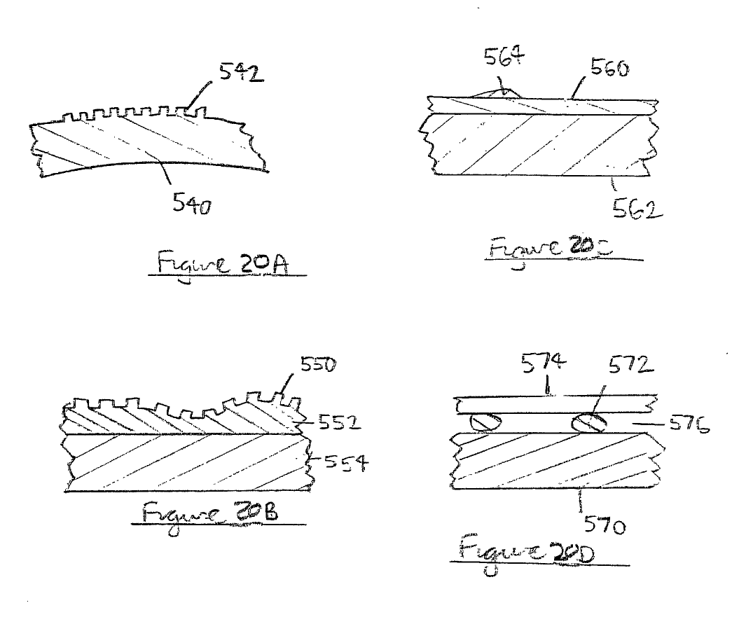

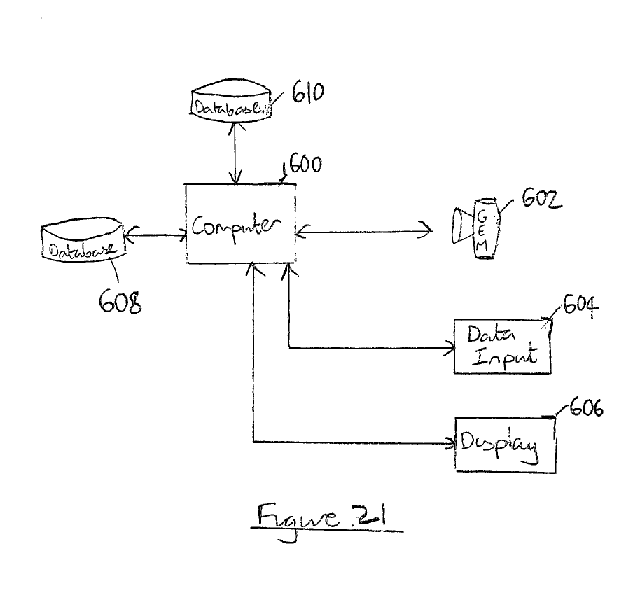

Referring to FIG. 22A, an example of a physical usage indicating means with a deformable element is illustrated. For example, a respiratory connector 622 contacts the port 624 of a respiratory analyzer 620, as previously described. As the connector 622 and port 624 are engaged, a crushable element 626 positioned therebetween is compressed. Preferably, the respiratory connector includes a notch 628 which engages with an end 630 of the port. FIG. 22B illustrates the port 624 after removal of the respiratory connector 622, for example after completion of a metabolic test, showing the crushable element 626 compressed. It should be appreciated that compression of a crushable element 626 may expose a warning message or symbol or the like at the surface 632 that discourages reuse. Referring to FIG. 22C, a warning message, such as “USED” as shown at 648, is exposed by compression of a crushable element 626 around the port 624 of the respiratory analyzer.

In another example, the crushable element assists in forming an airtight seal with the respiration connector, and the crushing process prevents reuse by preventing a subsequent good seal. In still another example, the end of the crushable element 626 and/or the connector port 622 is treated with adhesive, so that the crushable element is in whole or part pulled off the respiratory analyzer 620 after use. By damaging or removing the crushable element, lack of a good contact and sealing between the respiration connector and the port prevent or discourage future use.

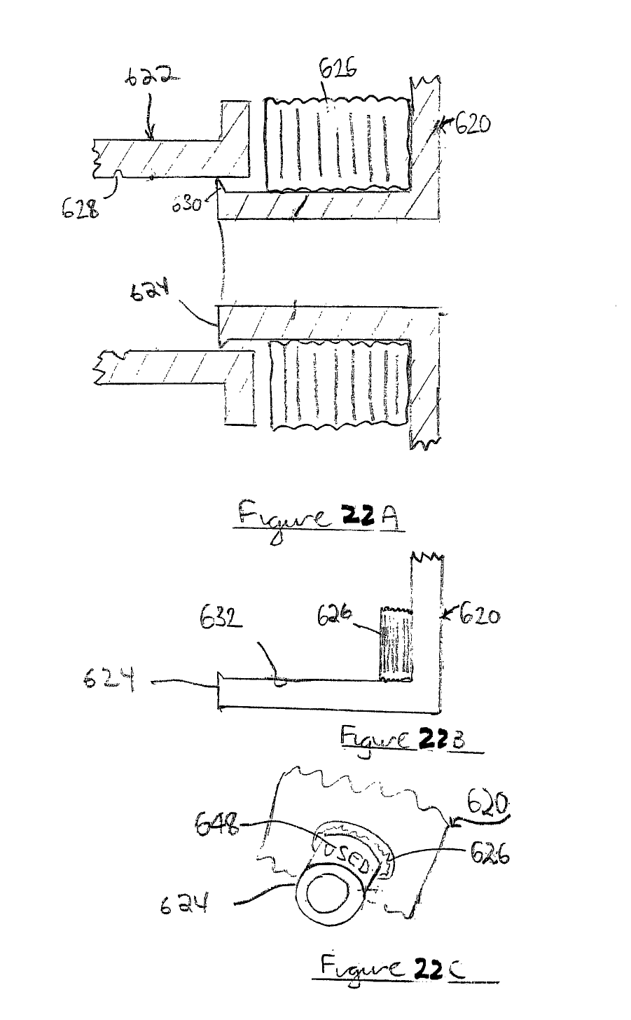

Referring to FIG. 23A, another example of a physical usage indicating means, which in this example is a peelable film, is illustrated. For example, aport 660 includes a peelable film 666 applied near the end, so that the respiratory connector 662 moves over the film 666. The film 666 assists in forming a good seal between the respiratory analysis system components. The respiratory connector 662 includes a lip, or hook, or other protrusion 664 which pushes over an edge of the film 666, as shown in FIG. 23B. In operation, as the respiratory connector is pulled away from the port 660, the lip 664 pulls the peelable film 666 off the surface of the port 660 as shown in FIG. 23C. It should be appreciated that the port is part of a respiratory analyzer, hygiene module, or other component.

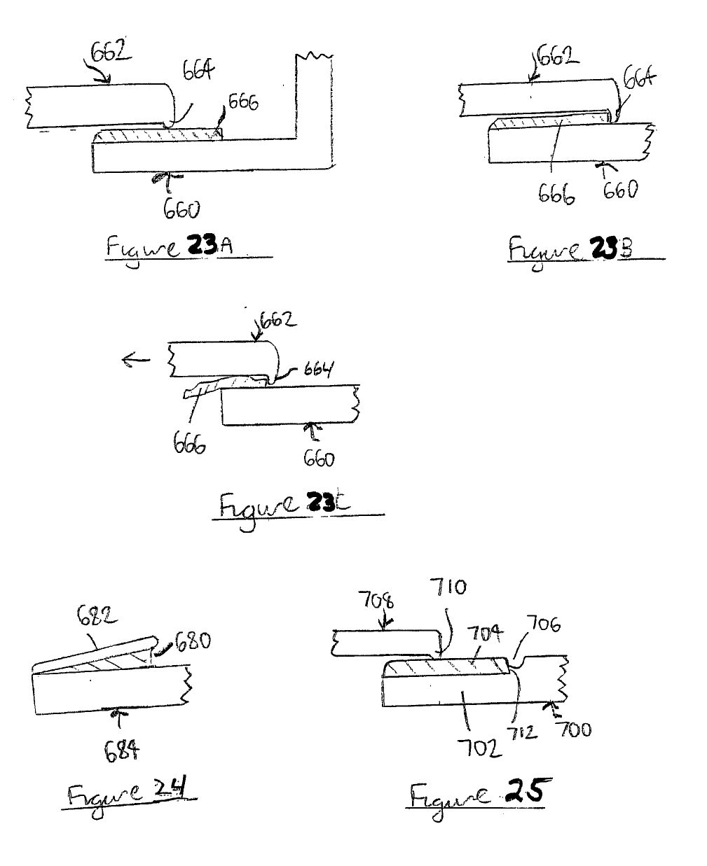

Referring to FIG. 24, still another example of a physical usage indicating means, which in this example is a resilient material 680 formed on the end of a port 684 covered with a surface layer 682, is illustrated. Characteristics of the surface layer are hardness, flexibility, and low friction. A respiration connector having a lip, as discussed above, is pushed over the surface layer. On removing the connector, the surface layer and resilient material are damaged as the connector is pulled away.

Referring to FIG. 25, yet another example of a physical usage indicating means, which in this example includes a port 700 having a main portion and an end portion 704, is illustrated. The respiratory connector 708 is pushed over the end portion. The step edge 712 of the main portion prevents the end portion from moving backwards, allowing a connector lip 710 to engage the depression 706. On removing the connector from the respiratory port, the end portion is pulled away from the port, preventing reuse or exposing a warning message to the user. Initially, the end portion can be weakly adhered to the port, or held on by friction, so that it is easily pulled away from the port. For example, the end portion 704 is a snap-on connector having a tab that is removed to disconnect the respirator connector from the respiratory analyzer.

Preferably, the removal of a film or other surface treatment exposes a warning symbol or message to the user. The removal occurs when a disposable element from a package, connecting a disposable element to a respiratory analyzer, or removing a disposable element from a respiratory analyzer.

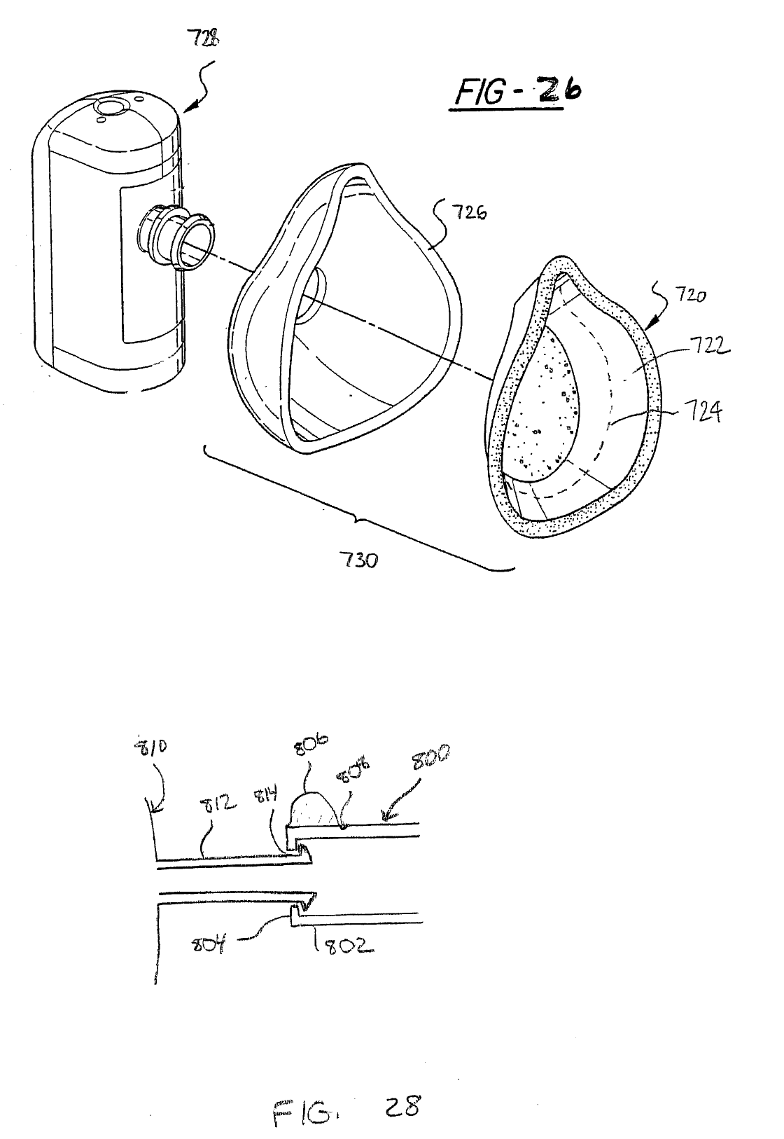

Referring to FIG. 26, another example of a physical usage indicating means, which in this example is a mask 730 having mask liner 720 disposed within a liner shell 722, is illustrated. The mask liner 720 includes a perforation, as shown at 724. As the mask liner 720 is removed from mask shell 726, which is attached to the respiratory analyzer 728, the mask shell separates due to the perforation 724, discouraging reuse. It should be appreciated that the mask shell 722 and liner 720 together form a mask 730.

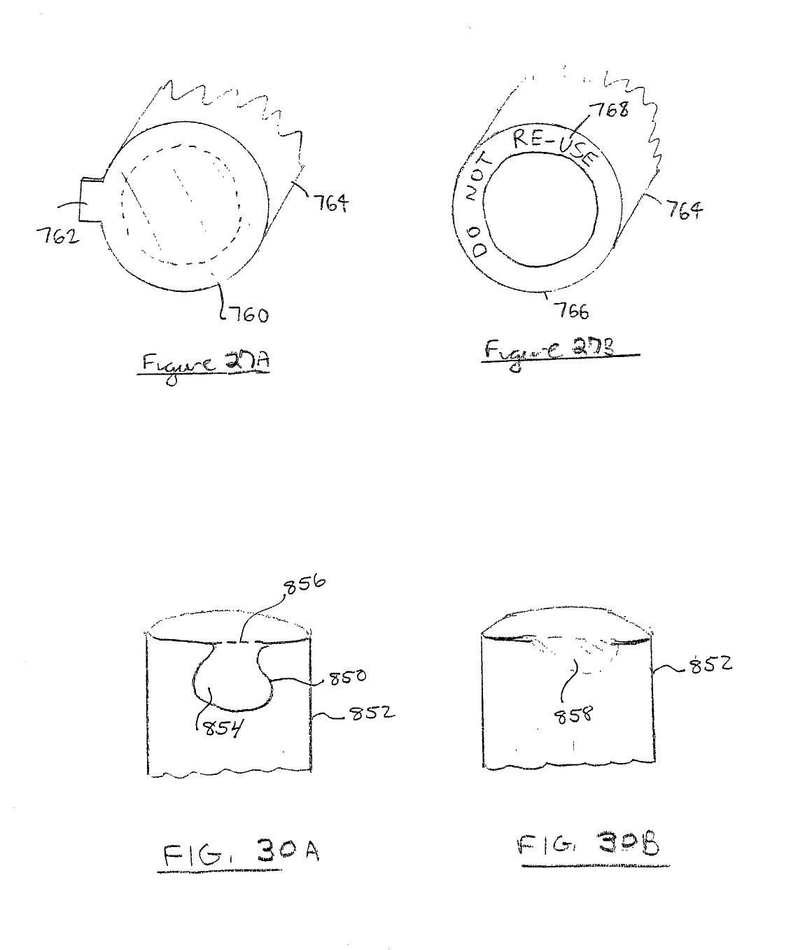

Referring to FIGS. 27A-72B, a further example of a physical usage indicating means is illustrated, which in this example is a removable cover 760. The removable cover 760 has a shape corresponding to the end of the respiratory connector. The removable cover is disposed over an end of the respiratory connector, to block a flow path through which respired gases pass. The removable cover 760 includes an outwardly extending tab 762. In use, the subject grips the tab 762 to remove the cover 760 from the end of the respiratory connector 764. This enables the subject to breathe through the flow path. Preferably, removal of the cover exposes a warning color, graphic, or other message, illustrated by the words “DO NOT REUSE”, as shown at 768 in FIG. 40B. It is contemplated that the removable cover 760 is positioned over an opening of a mask or mouthpiece. The removable cover 760 is fabricated from a material such as metal, plastic, metalized plastic, or the like.

Still a further example of physical indicating means is a packaging indicating means. For example, the packaging means is a package for the disposable element. Removal of the disposable element from the package necessitates the removal of a sticker, film, or the like, for example revealing a message not to use if the message was already displayed. In another example of a packaging means, a seal or film on the respiratory connector is broken by engaging a respiratory connector to a respiratory analyzer. Preferably, a message or warning not to reuse is displayed. In still another example of a packaging means, a disposable component, such as a mask, mouthpiece, filter, or filter module, is supplied sealed in a package with a desiccant. On removal from the package, ambient humidity changes the color of an indicator film, for example showing a warning to use once. Breathing through the disposable can accelerate the rate of color change. In yet another example of a packaging indicator means, the disposable component includes a perfumed scent, which dissipates when the packaging is opened. A person can be instructed only to use perfumed elements. Carbon dioxide in exhaled breath can induce an odor in a disposable component, discouraging reuse.

A further example of a physical usage indicating means is an indicator element, as previously described, with a predetermined life span. For example, a peak flow meter (not shown), as is known in the art, is used to determine the effectiveness of a filter element. It is known that pathogen filters become blocked over time, thus reducing the effectiveness of the filter and also reducing the accuracy of measurements due to obstruction of flow.

Using a peak flow meter, the subject is asked to exhale rapidly through the flow path, and the peak flow rate is determinable from the ultrasonic transducer signals. Assuming the person does not suffer from respiratory problems such as asthma, a low peak flow indicates a clogged filter and the need for replacement. An indicator such as an indicator light is illuminated. If the person does occasionally suffer from respiratory problems, the peak flow test can be valuable in establishing a suitable time for metabolic rate determination.

Another example of a physical usage indicating means with a predetermined lifespan is a transponder as shown at 770 in FIG. 18 is built into the mask that counts uses of the respiratory connector by receiving a signal from a transmitter as shown at 772 disposed within the main housing of the indirect calorimeter. Radiation can inductively couple with the transponder, providing power for the transponder, and other wireless signals can be used to increment or decrement a counter within the transponder module.

Still another example of a physical usage means with a predetermined lifespan is a usage sensor as shown at 774 of FIG. 18. The usage sensor is disposed within the respiratory analyzer, and senses the number of uses, such as respiratory tests performed. For an instrument primarily used by a single person, the person can be warned to change a disposable component after a certain number of uses. Yet another example of a usage means with a predetermined lifespan is an indicator element as shown at 776 of FIG. 18 on a disposable portion that fades over time, to encourage replacement. It should be appreciated that the fading may cause a message to be displayed.

A further example of a physical usage indicating means with a predetermined lifespan is a disposable portion provided with an identifying code, such as a barcode or other such code, as shown at 778 of FIG. 18. The code is entered into the respiratory analyzer before a test is performed. The respiratory analyzer is programmed to not perform a test if an acceptable identifying code is not supplied. A previously used code, or a code used more than a predetermined number of times, is not accepted, and the respiratory analyzer will not perform the test.

Still another example of a physical usage indicating means with a predetermined lifespan is a filter module as shown in FIGS. 15 and 16, that includes a filter material 448, or other material exposed to exhalations, that provides a visual indication of the presence of certain breath components, such as nitric oxide, ketones such as acetone, other volatile organic compounds, compounds indicative of oral bacteria, hydrogen, hydrogen sulfide, compounds indicative of bacteria in the stomach and intestinal tract, or other respiratory compounds. For example, an indication of ketones can indicate fat metabolism due to weight loss processes, or in other circumstances can indicate a metabolic disorder. Chemicals providing a colorimetric response to the presence of ketones and aldehydes in the breath can be supported by a filter material, in the form of particles, infusions into the filter, patches, films, and the like.

Another example of a physical usage means is a switch means positioned on the respiratory analyzer as shown at 780 in FIG. 28. The switch is in electrical communication with a usage control means 782, also in the respiratory analyzer. The switch 780 enables a predetermined number of uses of the respiratory connector. In use, the switch 780 is depressed, and the respiratory analyzer operates for a predetermined number of uses. The respiratory connector is removed and replaced after the predetermined number of uses, as controlled by the usage control means. Preferably, the number of uses is one and the switch 780 is combined with another usage indicating means to limit the number of uses. Alternatively, the switch means 780 is a one-way switch that is activated to remove the respiratory connector and also to attach the respiratory connector.

In still another example, to prevent users from bypassing the switch means, the switch means 780 includes a resistive element with a predetermined resistance, and the respiratory analyzer will only operate if the circuit is closed due to the presence of a corresponding resistive element with a predetermined resistance in the respiratory analyzer.

Yet another example of a physical usage indicating means is a sensing means shown at 784 of FIG. 15 in the non-disposable portion of the respiratory analyzer that detects the identity of a predetermined disposable portion. For example, the disposable respiratory connector can include an optimized coaxial flow path diameter for particular persons or activities. Preferably, the calculations performed by circuitry within the non-disposable part are modifiable by the parameters of the disposable, including cross-sectional area of the flow path, and dead space.

Referring to FIG. 28, a further example of a physical usage means is illustrated, which is a respiratory connector 800 with a detaching means that prevents reuse of the respiratory connector. For example, an open end of a port 802 in the respiratory connector 800 includes a radially extending breakaway rim 804. The rim also includes an outwardly extending tab 806. The port 802 may include a stress riser, such as a groove as shown at 808, at the junction of the tab 806 and rim 804. The respiratory analyzer 810 includes a port 812 with a groove 814 for receiving the rim 804 of the respiratory connector, to retain the respiratory connector 800 on the respiratory analyzer. To assemble the respiratory connector 800 to the respiratory analyzer 810, the respiratory analyzer port 802 slides over the respiratory analyzer port 812 until the rim 804 is engaged by the groove 814 in the respiratory analyzer port 812. To detach the respiratory connector 800 from the respiratory analyzer 810, the subject grips the tab 806 and pulls the tab 806 with a circular motion, thus removing the rim 804 of the respiratory connector port 802. The respiratory connector 800 slides off the respiratory analyzer 810. Advantageously, the respiratory connector 800 cannot be reused, since it will not be retained on the respiratory analyzer 810 without the rim 804. In addition, the presence of the rim 804 helps ensure a good seal between the respiratory connector port 802 and respiratory analyzer port 812, to prevent leaks.

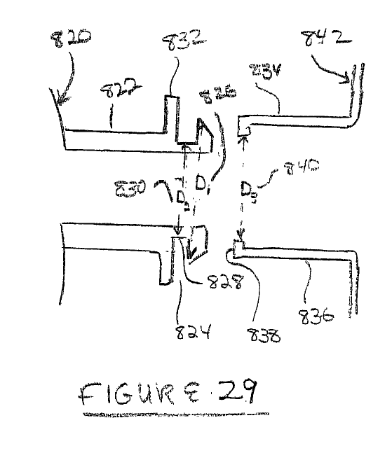

Referring to FIG. 29, still a further example of a physical usage means which prevents or limits reuse of the respiratory connector is illustrated. In this example, the respiratory connector 820 includes a respiratory port 822. An outer end 824 of the respiratory connector port 822 has a first diameter, D₁, shown at 826. The outer end of the respiratory connector port includes a groove 828 having a second diameter D₂, as shown at 830. The outer end 824 of the respiratory connector port 822 also includes an outwardly extending stop 832. The outer end 824 forms a deformable tab. An outer end 834 of a respiratory analyzer port 836 includes a radially extending lip 838, having a third diameter, shown at 840. It should be appreciated that D₂<D₃<D₁. To assemble the respiratory connector 820 to the respiratory analyzer 842, the respiratory analyzer port 834 slides over the respiratory connector port 822 until the lip 838 is retained in the groove 828. To remove the respiratory connector 820, the respiratory connector 820 is pulled off the respiratory analyzer port 834, thus deforming the end tab 824 to prevent reuse of the respiratory connector 820.

Referring to FIGS. 30A and 30B, another example of a physical indicator element, which is a tear strip 850, is illustrated. The tear strip is integral with the respiratory connector 852, and includes a tab 854. The junction of the tear strip 850 and respiratory connector 852 includes a stress riser as shown at 856, such as a perforation, or thinner area of material, or the like. In use, the tear strip 850 is torn off to remove the respiratory connector 852 from the respiratory analyzer, thus removing a weakened section of the respiratory connector, as shown at 858, to prevent reuse of the respiratory connector 852.

The present invention has been described in an illustrative manner. It is to be understood that the terminology, which has been used, is intended to be in the nature of words of description rather than of limitation.

Many modifications and variations of the present invention are possible in light of the above teachings. Therefore, within the scope of the appended claims, the present invention may be practiced other than as specifically described.

1. A respiratory connector for passing inhaled and exhaled gases as a subject breathes into a respiratory analyzer comprising:
a housing configured to be supported in contact with the subject;
a flow pathway within the housing for passing the inhaled and exhaled gases therethrough;
a connector port extending from the housing for connecting the respiratory connector to the respiratory analyzer; and
a usage indicating means within the housing for indicating usage of the respiratory connector to the subject.

2. The respiratory connector according to claim 1 wherein said usage indicating means is a visual indicator of previous usage of the respiratory connector.

3. The respiratory connector according to claim 2 wherein said visual indicator is a colorimetric indicator that undergoes a colorimetric change when exposed to a predetermined condition.

4. The respiratory connector according to claim 3 wherein said colorimetric indicator is a colorimetric film.

5. The respiratory connector according to claim 3 wherein said colorimetric indicator is dispersed throughout said respiratory connector, so that said respiratory connector changes colors when exposed to a predetermined condition.

6. The respiratory connector according to claim 3 wherein said colorimetric indicator is arranged in a predetermined pattern that indicates a word upon the occurrence of a predetermined condition.

7. The respiratory connector according to claim 3 wherein said colorimetric indicator is a moisture sensitive film that indicates a word when exposed to moisture in the inhaled and exhaled breath of the subject.

8. The respiratory connector according to claim 3 wherein said colorimetric indicator changes color when exposed to a predetermined temperature.

9. The respiratory connector according to claim 2 wherein said visual indicator is a pressure-induced distortion that occurs when said pressure sensitive visual usage indicator is exposed to a predetermined condition.

10. The respiratory connector according to claim 1 wherein said usage indicator is a usage identifying means for identifying the respiratory connector and if the respiratory connector has been previously used by the respiratory analyzer.

11. The respiratory connector according to claim 1 wherein said usage indicating means is a physical usage indicating means for indicating previous usage of the respiratory connector.

12. The respiratory connector according to claim 11 wherein said physical usage indicating means is a deformable member disposed between a respiratory analyzer connector port and the respiratory connector port, wherein said deformable member deforms as the respiratory connector interconnects with the respiratory analyzer.

13. The respiratory connector according to claim 11 wherein said physical usage indicating means is a cover with an integral tab disposed over an open end of the respiratory connector port, wherein said cover is removed prior to use of the respiratory connector.

14. The respiratory connector according to claim 11 wherein said physical usage indicating means is a removable tear strip integral with the respiratory connector for tearing off a portion of the respiratory connector port, such that the tear strip is removed before disconnecting the respiratory connector from the respiratory analyzer.

15. The respiratory connector according to claim 11 wherein said physical usage indicating means is a detachable member integrally formed in an open end of the connecting port of said respiratory connector, wherein said open end includes a detachable rim that is engaged within a groove in the respiratory analyzer port when the respiratory connector and respiratory analyzer are interconnected, and is detached to disconnect the respiratory connector from the respiratory analyzer.

16. The respiratory connector according to claim 11 wherein said physical indicating means is a deformable tab integrally formed in a connecting end of said respiratory connector that is deformed by disconnecting the respiratory connector from the respiratory analyzer.

17. The respiratory connector as set forth in claim 13 wherein said physical usage indicating means is a peelable film disposed on the connector port, such that the film is peeled away as the respiratory connector is detached from the respiratory analyzer.

18. The respiratory connector as set forth in claim 13 wherein said physical usage indicating means is a packaging means for packaging said respiratory connector that indicates no previous usage of the respiratory connector.

19. The respiratory connector according to claim 11 wherein said physical usage indicating means is an indicator element having a counter for counting each use of the respiratory connector so that additional use of the respiratory connector is prevented after a predetermined number of uses.

20. The respiratory connector according to claim 1 wherein said physical usage indicating means is a sensor for sensing use of the respiratory connector so that additional use of the respiratory connector is prevented after a predetermined number of uses.

21. A respiratory connector for passing inhaled and exhaled gases as a subject breathes into a respiratory analyzer comprising:
an outer shell having a generally hemispherical shape, with an opening in the outer shell in fluid communication with a flow path for the respiratory connector;
a removable shell liner inserted in said outer shell, wherein said shell liner includes a hygiene barrier positioned over the opening in said outer shell for operatively passing the inhaled and exhaled gases therethrough while blocking a predetermined pathogen in the exhaled gas; and
a usage indicating means within the respiratory connector for indicating usage of the respiratory connector to the subject.

22. The respiratory connector according to claim 21 wherein said shell liner includes a generally planar face seal having an opening for the mouth and nose of the subject, for sealing the respiratory connector to the face of the user, a liner wall extending from an outer edge of the face seal and generally perpendicular to the face seal, and said hygiene barrier extends therebetween a free edge of the liner wall and opposing the opening in the face seal.

23. The respiratory connector according to claim 22 wherein said usage indicator is pressure sensitive visual usage indicator disposed on a surface of the face seal that distorts the surface of the face seal when exposed to a predetermined condition.

24. The respiratory connector according to claim 21 wherein said hygiene barrier includes a colorimetric usage indicator that changes color from exposure to a predetermined condition.

25. The respiratory connector according to claim 21 wherein said outer shell includes a colorimetric usage indicator that changes color from exposure to a predetermined condition.

26. The respiratory connector according to claim 21 wherein said physical usage indicator is a separating means for separating said mask liner into a plurality of sections as the mask liner is removed from the mask shell.

27. The respiratory connector according to claim 21 wherein said usage indicator is a colorimetric usage indicator is arranged in a predetermined pattern that indicates a word upon the occurrence of a predetermined condition.

28. The respiratory connector according to claim 21 wherein said usage indicator is a colorimetric indicator that changes color when exposed to a predetermined temperature.

29. The respiratory connector according to claim 21 wherein said usage indicating means is a physical usage indicating means for indicating previous usage of the respiratory connector.

30. A respiratory connector for passing inhaled and exhaled gases as a subject breathes into a respiratory analyzer comprising:
a mouthpiece;
a hygiene barrier module having an inlet port for operatively connecting said hygiene barrier module to said mouthpiece, such that the inhaled and exhaled gases pass therethrough said hygiene barrier module for blocking a predetermined pathogen from the exhaled gases and a connecting port for operatively connecting the respiratory connector to a corresponding inlet conduit for the respiratory analyzer; and
a usage indicating means within the respiratory connector for indicating usage of the respiratory connector to the subject.

31. The respiratory connector according to claim 30 wherein said hygiene barrier includes a colorimetric usage indicator that changes color from exposure to a predetermined condition.

32. The respiratory connector according to claim 30 wherein said mouthpiece includes a colorimetric usage indicator that changes color from exposure to a predetermined condition.

33. The respiratory connector according to claim 30 wherein said usage indicator is a colorimetric usage indicator is arranged in a predetermined pattern that indicates a word upon the occurrence of a predetermined condition.

34. The respiratory connector according to claim 30 wherein said usage indicating means is a physical usage indicating means for indicating previous usage of the respiratory connector.
